(19) United States
(12) Patent Application Publication
(10) Pub. No.: US 2007/0219193 A1
(43) Pub. Date: Sep. 20, 2007

(54) ALKYLAMINE-SUBSTITUTED BICYCLIC ARYL COMPOUNDS USEFUL AS MODULATORS OF PPAR

(75) Inventor: Cunxiang Zhao, San Diego, CA (US)

Correspondence Address: GLOBAL PATENT GROUP;ATTN: MS LAVERN HALL, 10411 Clayton Road, Suite 304, Frontenac, MO, 63131, US

(73) Assignee: KALYPSYS, INC, San Diego, CA (US)

(21) Appl. No.: 11/687,485

(22) Filed: Mar. 16, 2007

Related U.S. Application Data
(60) Provisional application No. 60/783,709, filed on Mar. 17, 2006.

(51) Int. Cl. A61K 31/538; A61K 31/517; A61K 31/506; C07D 413/02; C07D 403/02

(52) U.S. Cl. 514/230.5; 544/105; 544/284; 544/295; 514/275; 514/266.21; 514/249

(57) ABSTRACT

The present invention relates to novel alkylamine-substituted bicyclic aryl compounds, pharmaceutical compositions comprising the same, useful as modulators of PPAR, and methods for the treatment or prevention of disease.

This application claims the benefit of priority of U.S. provisional application No. 60/783,709, filed Mar. 17, 2006, the disclosure of which is hereby incorporated by reference as if written herein in its entirety.

### FIELD OF THE INVENTION

The present invention relates to novel alkylamine-substituted bicyclic aryl derivatives and methods for treating various diseases by modulation of nuclear receptor mediated processes using these compounds, and in particular processes mediated by peroxisome proliferator activated receptors (PPARs).

### BACKGROUND OF THE INVENTION

Peroxisome proliferators are a structurally diverse group of compounds which, when administered to mammals, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidemic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Subtypes of PPAR include PPAR-alpha, PPAR-delta (also known as NUC1, PPAR-beta and FAAR) and two isoforms of PPAR-gamma. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPAR-alpha (or alternatively, PPARα), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J. 11:433-439 (1992); Bardot et al., Biochem. Biophys. Res. Comm. 192:37-45 (1993); Muerhoff et al., J. Biol. Chem. 267:19051-19053 (1992); and Marcus et al., Proc. Natl. Acad. Sci. USA 90(12):5723-5727 (1993).

Activators of the nuclear receptor PPAR-gamma (or alternatively, PPARγ), for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

Transgenic expression of an activated form of PPAR-delta (or alternatively, PPARδ, PPARβ, or NUC1) in adipose tissue produces lean mice that are resistant to obesity, hyperlipidemia and tissue steatosis induced genetically or by a high-fat diet. The activated receptor induces genes required for fatty acid catabolism and adaptive thermogenesis. Interestingly, the transcription of PPAR-γ target genes for lipid storage and lipogenesis remain unchanged. In parallel, PPAR-δ deficient mice challenged with a high-fat diet show reduced energy uncoupling and are prone to obesity.

PPARδ has been shown to be a valuable molecular target for treatment of dyslipidemia and other diseases. For example, in a recent study in insulin-resistant obese rhesus monkeys, a potent and selective PPARδ compound was shown to decrease VLDL and increase HDL in a dose response manner (Oliver et al., Proc. Natl. Acad. Sci. U.S.A. 98: 5305, 2001). Also, in a recent study in wild-type and HDL-lacking, ABCA1^{-/-} mice, a different potent and selective PPARδ compound was shown to reduce fractional cholesterol absorption in the intestine, and coincidentally reduce expression of the cholesterol-absorption protein NPC1L1 (van der Veen et al., J. Lipid Res. 2005 46: 526-534).

Because there are three isoforms of PPAR and all of them have been shown to play important roles in energy homeostasis and other important biological processes in human body and have been shown to be important molecular targets for treatment of metabolic and other diseases (see Wilson, et al. J. Med. Chem. 43: 527-550 (2000)), it is desired in the art to identify compounds which are capable of interacting with multiple PPAR isoforms or compounds which are capable of selectively interacting with only one of the PPAR isoforms. Such compounds would find a wide variety of uses, such as, for example, in the treatment or prevention of obesity, for the treatment or prevention of diabetes, dyslipidemia, metabolic syndrome X and other uses.

Several PPAR-modulating drugs have been approved for use in humans. Fenofibrate and gemfibrozil are PPARα modulators; pioglitazone (Actos, Takeda Pharmaceuticals and Eli Lilly) and rosiglitazone (Avandia, GlaxoSmithKline) are PPARγ modulators. All of these compounds have liabilities as potential carcinogens, however, having been demonstrated to have proliferative effects leading to cancers of various types (colon; bladder with PPARα modulators and liver with PPARγ modulators) in rodent studies. Therefore, a need exists to identify modulators of PPARs that lack these liabilities.

### SUMMARY OF THE INVENTION

Novel compounds and pharmaceutical compositions that ameliorate metabolic disorders by modulating PPAR have been found, together with methods of synthesizing and using the compounds including methods for modulating PPAR in a patient by administering the compounds.

The present invention discloses a class of compounds, useful in treating PPAR-mediated disorders and conditions, defined by structural Formula I:

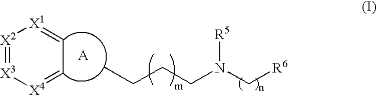

Or a salt, ester, or prodrug thereof, wherein:

A is selected from the group consisting of cycloalkyl and heterocycloalkyl, which may be optionally substituted;

X¹ is selected from the group consisting of CR¹ and N;

X² is selected from the group consisting of CR² and N;

X³ is selected from the group consisting of CR³ and N;

X⁴ is selected from the group consisting of CR⁴ and N; or any two of X¹, X², X³ and X⁴ may combine to form aryl, cycloalkyl or heterocycloalkyl, any of which may be optionally substituted;

m is 0, 1 or 2;

n is 0, 1, 2 or 3;

R¹-R⁴ are independently selected from the group consisting of alkoxy, alkyl, aryl, arylalkyl, carboxyalkyl, cycloalkyl, esteralkyl, halo, haloalkyl, heteroarylalkyl, heterocycloalkyl, heterocycloalkylalkyl and hydrogen, any of which may be optionally substituted; or any two of R¹, R², R³, and R⁴ may combine to form aryl, cycloalkyl and heterocycloalkyl, which may be optionally substituted; and

R⁵ and R⁶ are independently selected from the group consisting of acyl, alkyl, alkoxy, alkoxyalkyl, alkylene, alkynyl, amido, amino, aminosulfonyl, aryl, arylalkoxy, arylamino, arylthio, carboxy, cycloalkyl, ester, ether, halo, haloalkyl, heteroaryl, heteroarylamino, heterocycloalkyl, hydrazinyl, imino, thio, sulfonate and sulfonyl, any of which may be optionally substituted.

Compounds according to the present invention possess useful PPAR modulating activity, and may be used in the treatment or prophylaxis of a disease or condition in which PPAR plays an active role. Thus, in broad aspect, the present invention also provides pharmaceutical compositions comprising one or more compounds of the present invention together with a pharmaceutically acceptable carrier, as well as methods of making and using the compounds and compositions. In certain embodiments, the present invention provides methods for modulating PPAR. In other embodiments, the present invention provides methods for treating a PPAR-mediated disorder in a patient in need of such treatment comprising administering to said patient a therapeutically effective amount of a compound or composition according to the present invention. The present invention also contemplates the use of compounds disclosed herein for use in the manufacture of a medicament for the treatment of a disease or condition ameliorated by the modulation of PPAR.

### DETAILED DESCRIPTION OF THE INVENTION

In certain embodiments, the compounds of the present invention have structural Formula II:

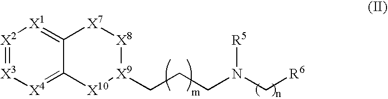

or a salt, ester, or prodrug thereof, wherein:

X¹ is selected from the group consisting of CR¹ and N;

X² is selected from the group consisting of CR² and N;

X³ is selected from the group consisting of CR³ and N;

X⁴ is selected from the group consisting of CR⁴ and N;

X⁷ is selected from the group consisting of C(O), CR^{7a}R^{7b}, O, NR⁷ and S(O)_{g};

X⁸ is selected from the group consisting of C(O), CR^{8a}R^{8b}, O, NR⁸ and S(O)_{g};

X⁹ is selected from the group consisting of CR^{9a} and N;

X¹⁰ is selected from the group consisting of C(O), CR^{10a}R^{10b}, O, NR¹⁰ and S(O)_{g};

m is 0, 1 or 2;

n is 0, 1, 2 or 3;

g is 0, 1 or 2;

R⁵ and R⁶ are independently selected from the group consisting of aryl and heteroaryl, any of which may be optionally substituted;

R¹-R⁴ are independently selected from the group consisting of alkoxy, alkyl, alkylcarboxy, alkylester, alkylaryl, amido, carboxy, carboxyalkyl, halo, heteroaryl, heteroarylalkyl, heterocycloalkyl and hydrogen, any of which may be optionally substituted;

R^{7a}-R^{10a} and R^{7b}-R^{10b} are independently selected from the group consisting of alkoxy, alkyl, aryl, alkylaryl, carboxy, cycloalkyl, cyano, ester, halo, haloalkyl, heteroarylalkyl, heterocycloalkyl, hydrogen and hydroxyl, any of which may be optionally substituted; and

R⁷-R¹⁰ are independently selected from the group consisting of alkyl, alkylaryl, aryl, cycloalkyl, halo, haloalkyl, heteroaryl, heterocycloalkyl and hydrogen, any of which may be optionally substituted.

In further embodiments, compounds of the present invention have structural Formula III:

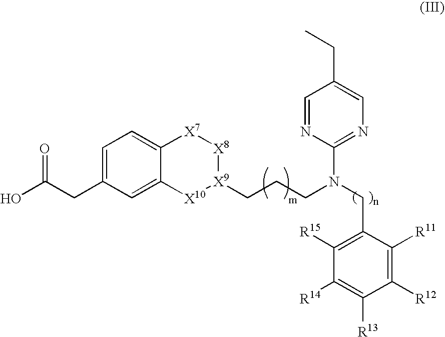

X⁷ is selected from the group consisting of CR^{7a}R^{7b}, O, and NR⁷;

X⁸ is selected from the group consisting of CR^{7a}R^{8b}, O, and NR⁸;

X⁹ is selected from the group consisting of CR^{9a} and N;

X¹⁰ is selected from the group consisting of CR^{10a}R^{10b}, O, and NR¹⁰;

m is 0, 1 or 2;

n is 0, 1 or 2;

R^{7a}-R^{10a} and R^{7b}-R^{10b} are independently selected from the group consisting of alkoxy, alkyl, halo, hydrogen and hydroxyl, any of which may be optionally substituted;

R⁷-R¹⁰ are independently selected from the group consisting of alkyl, haloalkyl, hydrogen and null, any of which may be optionally substituted; and

R¹¹, R¹², R¹³, R¹⁴ and R¹⁵ are independently selected from the group consisting of alkoxy, alkyl, halo, haloalkyl and hydrogen, any of which may be optionally substituted.

In certain embodiments, the invention provides for compounds wherein X⁷ is CR^{7a}R^{7b} and X⁸ is CR^{8a}R^{8b}.

In further embodiments, X⁷ and X⁸ are each CH₂.

In further embodiments, compounds of the present invention have structural Formula IV:

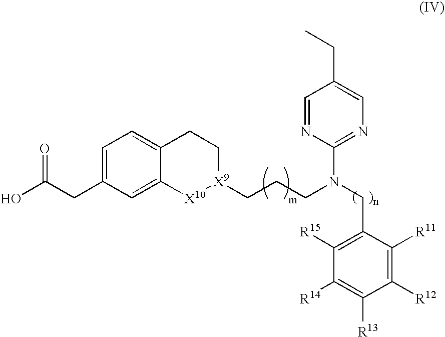

wherein:

X⁹ is selected from the group consisting of CH or N;

X¹⁰ is selected from the group consisting of CH₂ or O;

m is 0, 1 or 2;

n is 0, 1 or 2; and

R¹¹-R¹⁵ are independently selected from the group consisting of alkoxy, alkyl halo, haloalkyl and hydrogen, any of which may be optionally substituted.

In further embodiments, X⁹ is N and X¹⁰ is CH₂.

In other embodiments, X⁹ is CH and X¹⁰ is O.

In yet further embodiments, R¹³ is selected from the group consisting of trifluoromethyl and trifluoromethoxy; and R¹¹, R¹², R¹⁴, and R¹⁵ are hydrogen.

In other embodiments, compounds of the present invention have structural Formula V

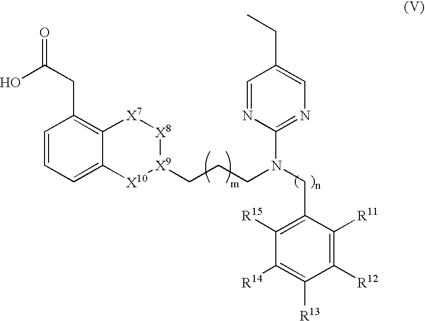

X⁷ is selected from the group consisting of CR^{7a}R^{7b}, O, and NR⁷;

X⁸ is selected from the group consisting of CR^{8a}R^{8b}, O, and NR⁸;

X⁹ is selected from the group consisting of CR^{9a} and N;

X¹⁰ is selected from the group consisting of CR^{10a}R^{10b}, O, and NR¹⁰;

m is 0, 1 or 2;

n is 0, 1 or 2;

R^{7a}-R^{10a} and R^{7b}-R^{10b} are independently selected from the group consisting of alkoxy, alkyl, halo, hydrogen and hydroxyl, any of which may be optionally substituted;

R⁷-R¹⁰ are independently selected from the group consisting of alkyl, haloalkyl, hydrogen and null, any of which may be optionally substituted; and

R¹¹, R¹², R¹³, R¹⁴ and R¹⁵ are independently selected from the group consisting of alkoxy, alkyl, halo, haloalkyl and hydrogen, any of which may be optionally substituted.

Further embodiments are afforded by Formula V to include compounds wherein X⁷ and X⁸ are CH₂, X⁹ is CH and X¹⁰ is oxygen.

Yet further embodiments are afforded by Formula V to include compounds wherein X⁷ and X¹⁰ are CH₂, X⁹ is CH and X⁸ is oxygen.

In further embodiments, X⁷ is CR^{7a}R^{7b} and X⁹ is CR^{9a}.

In yet further embodiments, X⁷ is CH₂ and X⁹ is CH.

### In further embodiments, compounds of the present invention have structural Formula VI

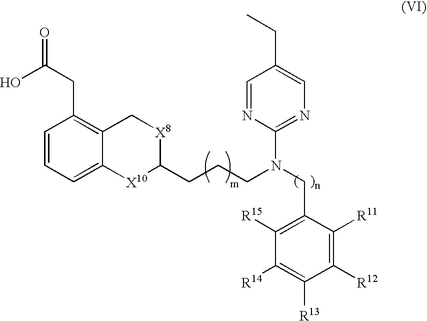

X⁸ and X¹⁰ are each independently selected from the group consisting of CH₂ or O;

m is 0, 1 or 2;

n is 0, 1 or 2; and

R¹¹-R¹⁵ are independently selected from the group consisting of alkoxy, alkyl halo, haloalkyl and hydrogen, any of which may be optionally substituted.

In further embodiments, X⁸ is O and X¹⁰ is CH₂.

In other embodiments, X⁸ is CH₂ and X¹⁰ is O.

In yet further embodiments, R¹³ is selected from the group consisting of trifluoromethyl and trifluoromethoxy, and R¹¹, R¹², R¹⁴, and R¹⁵ are hydrogen.

In certain embodiments, the compound is selected from the group consisting of Examples 1-17, 18a -18d, and 19a -19d.

The compounds disclosed herein may also be used in the manufacture of a medicament for the prevention or treatment of a disease or condition ameliorated by the modulation of PPAR.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(≡O)—NR₂ group with R as defined herein. The term “N-amido” as used herein, alone or in combination, refers to a RC(≡O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to—NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2, 1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′—group with R and R′ as defined herein.

The term “S-sulfonamido” refers to a —S(═O)₂NRR′, group, with R and R′ as defined herein.

The terms “thia” and “thio,” as used herein, alone or in combination, refer to a S group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR group with X is a halogen and R as defined herein.

The term “trihalomethanesulfonyl” refers to a X₃CS(O)₂ group where X is a halogen.

The term “trihalomethoxy” refers to a X₃CO group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

PPAR modulator is used herein to refer to a compound that exhibits an EC₅₀ with respect to PPAR activity of no more than about 100 μM and more typically not more than about 50 μM, as measured in the PPAR transcriptional assays described generally hereinbelow. EC₅₀ is that concentration of modulator which either activates or reduces the activity of an enzyme (e.g., PPAR) to half-maximal level. Representative compounds of the present invention have been discovered to exhibit modulatory activity against PPAR. Compounds of the present invention preferably exhibit an EC₅₀ with respect to PPAR of no more than about 10 μM, more preferably, no more than about 5 μM, even more preferably not more than about 1 μM, and most preferably, not more than about 200 nM, as measured in the PPAR assay(s) described herein.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, dectuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5°% w/w, more preferably from 0.1% to 1% w/w of the formulation.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with: (a) statin and/or other lipid lowering drugs for example MTP inhibitors and LDLR upregulators; (b) antidiabetic agents, e.g. metformin, sulfonylureas, or PPAR-gamma, PPAR-alpha and PPAR-alpha/gamma modulators (for example thiazolidinediones such as e.g. Pioglitazone and Rosiglitazone); and (c) antihypertensive agents such as angiotensin antagonists, e.g., telmisartan, calcium channel antagonists, e.g. lacidipine and ACE inhibitors, e.g., enalapril.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating PPAR-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of PPAR-mediated disorders.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

### The following schemes can be used to practice the present invention.

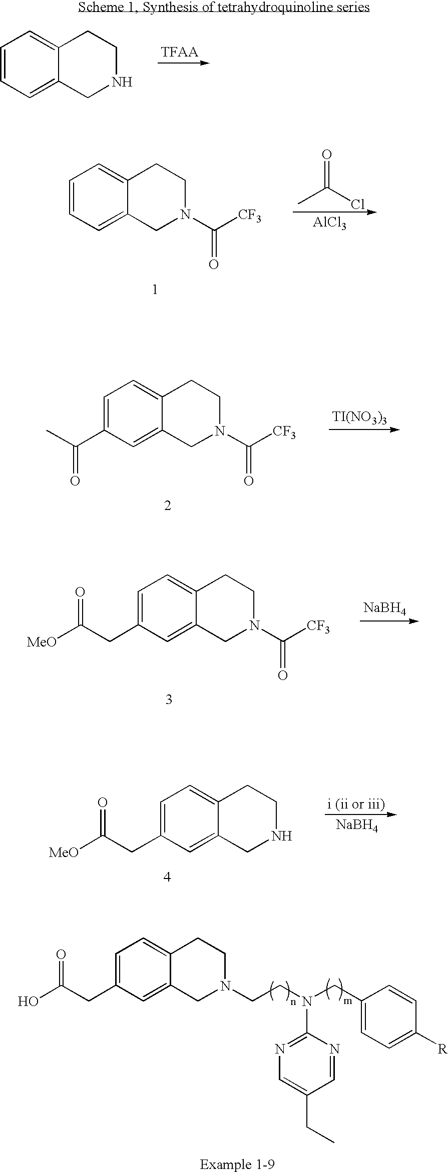

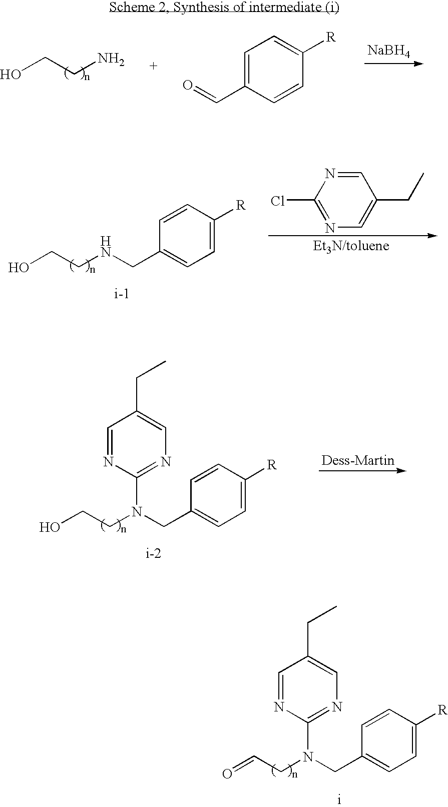

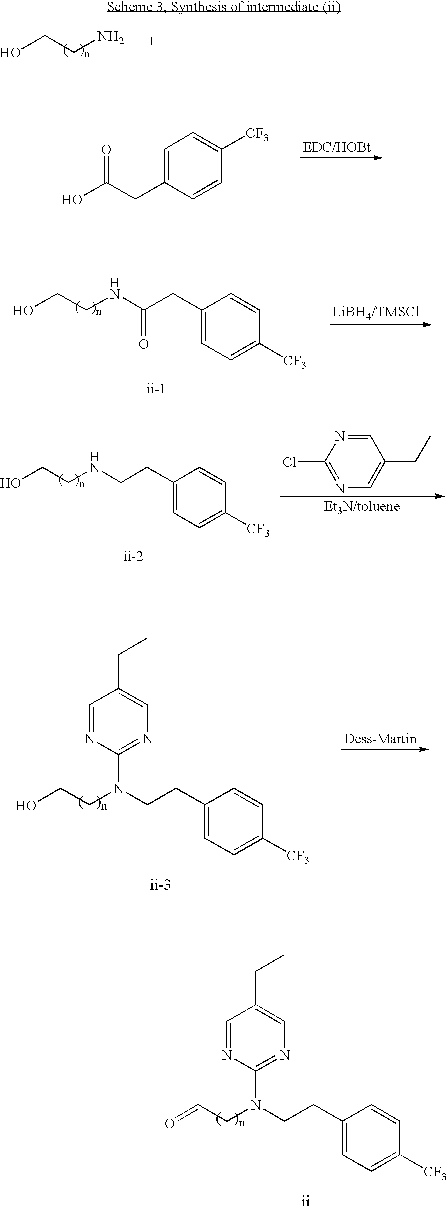

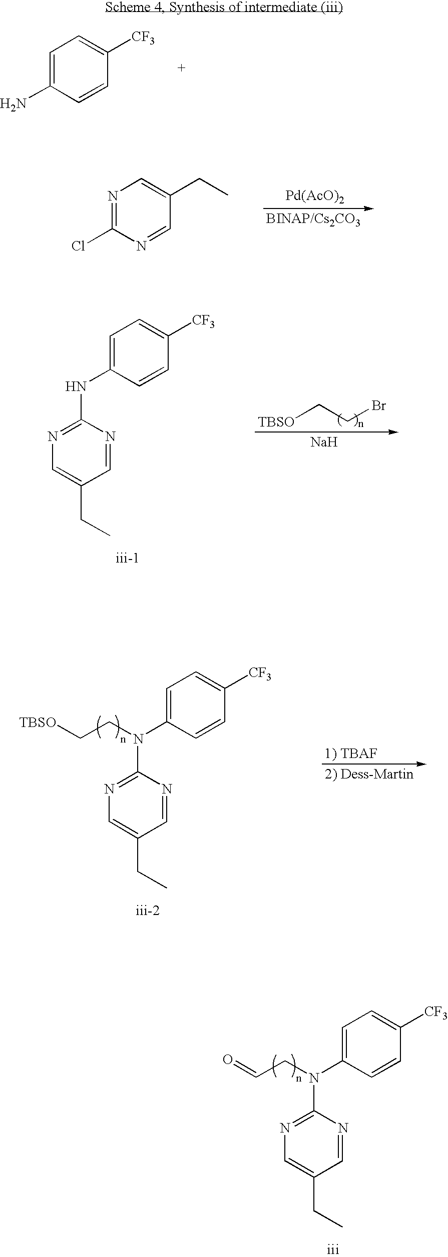

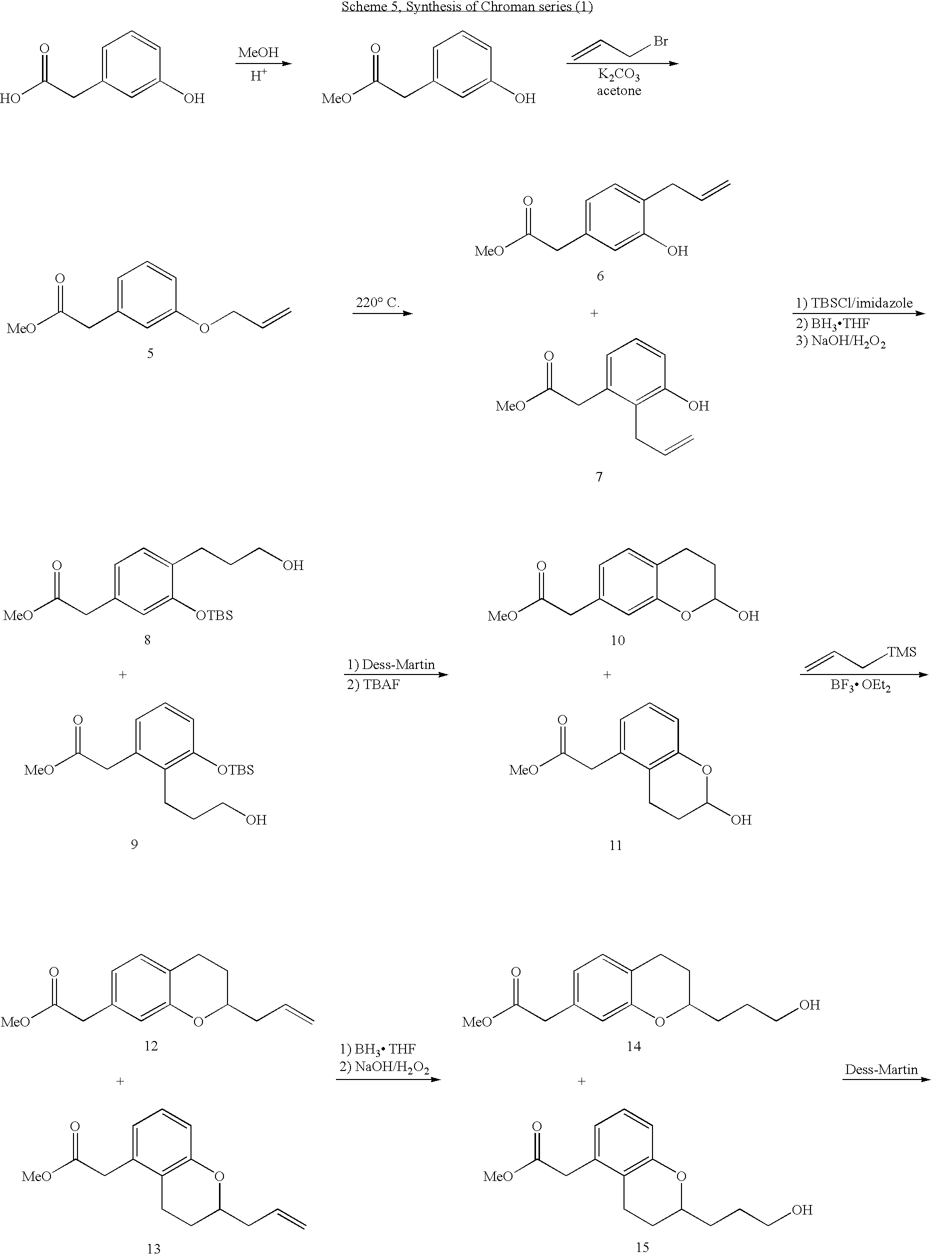

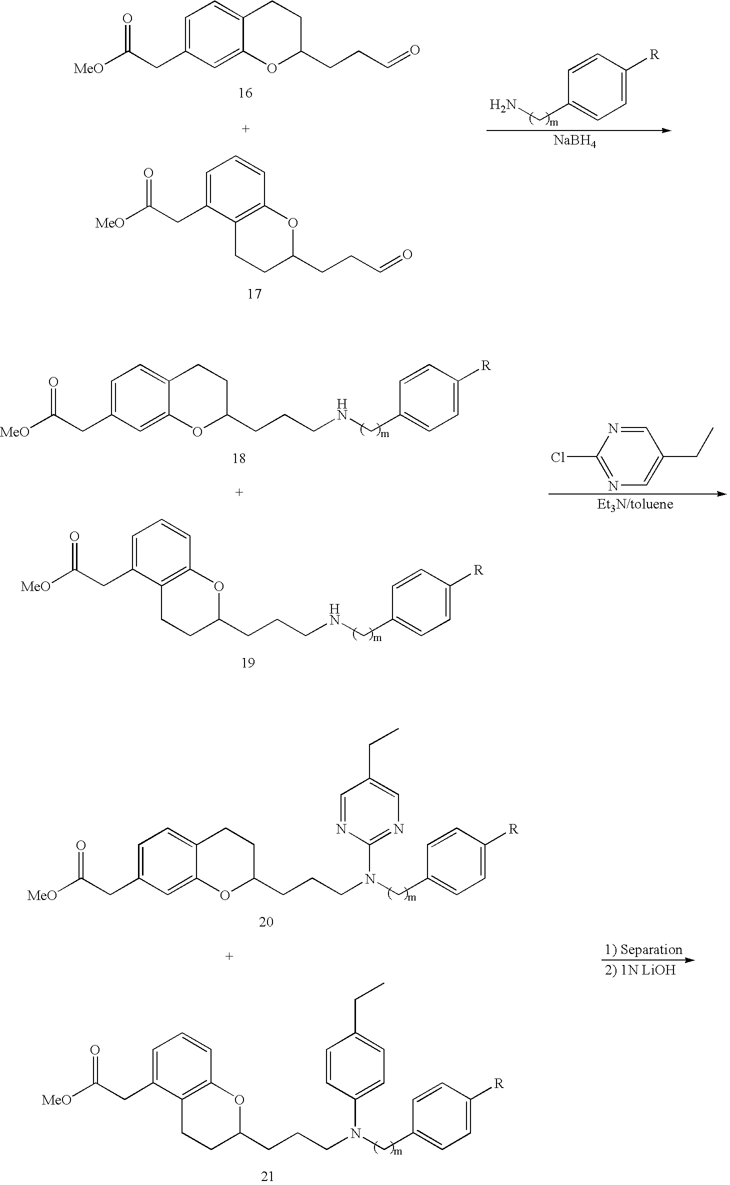

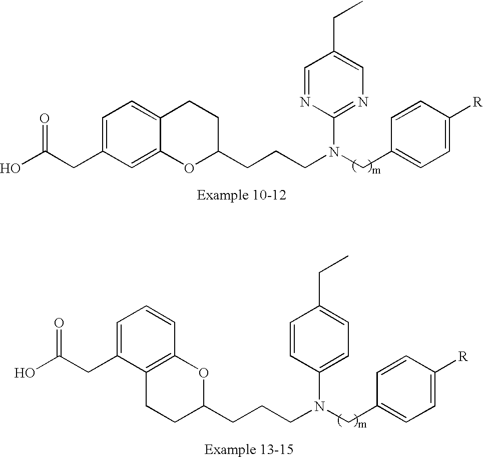

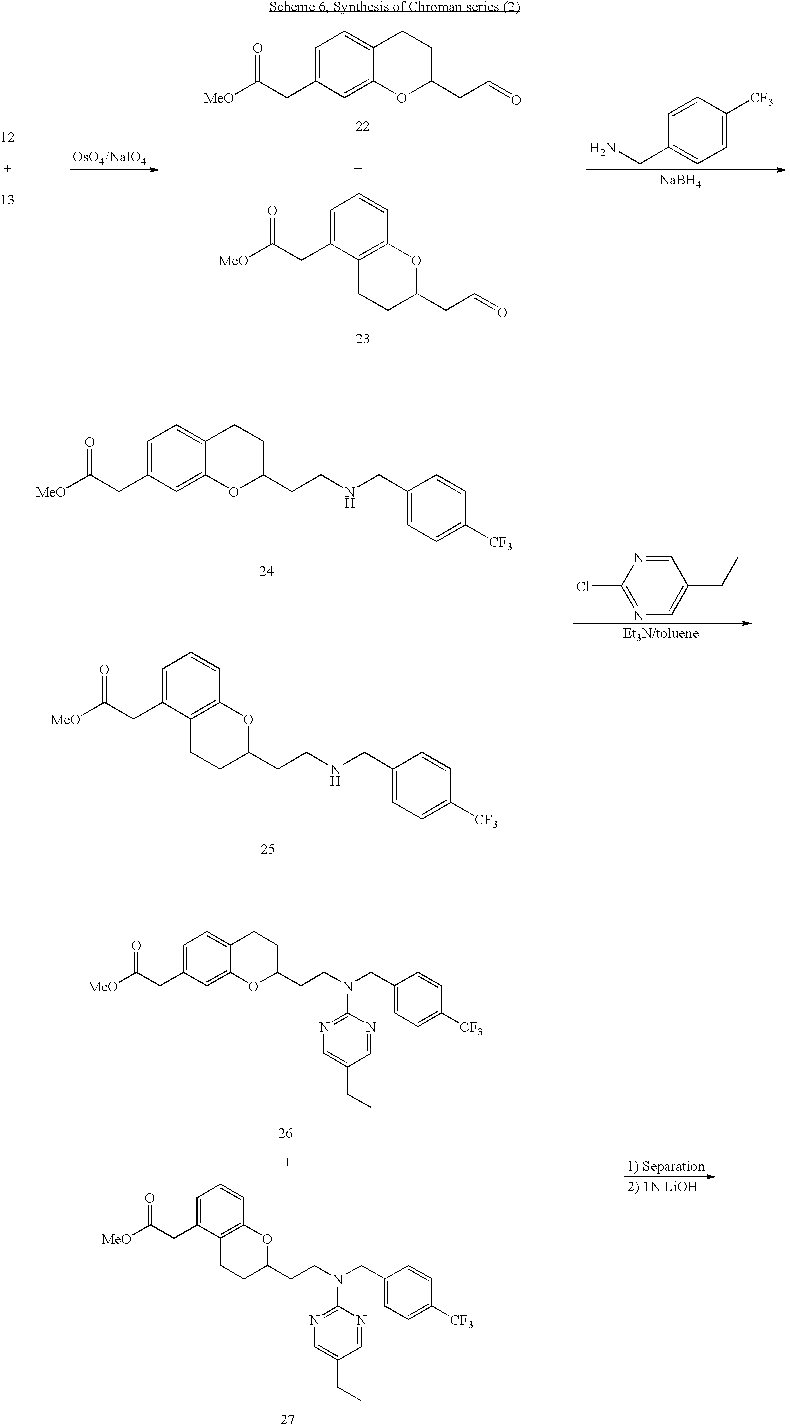

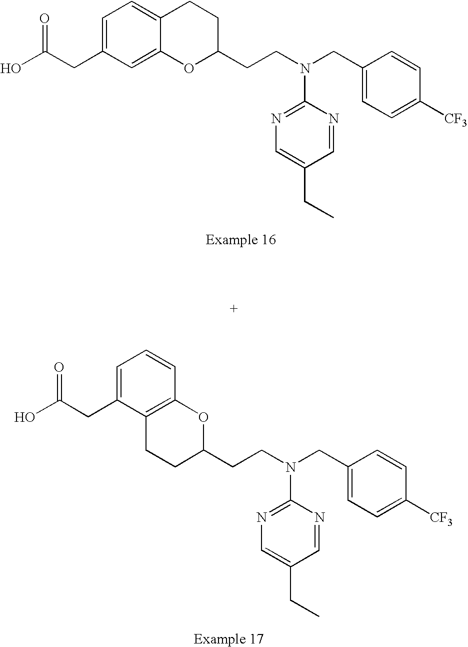

The invention is further illustrated by the following examples. IUPAC names for compounds and intermediates may have been generated using CambridgeSoft's ChemDraw 10.0.

### EXAMPLE 1

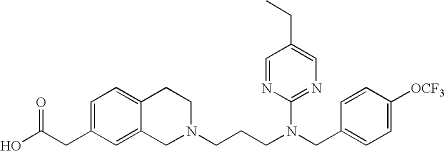

### (2-{3-[(5-Ethyl-pyrimidin-2-yl)-(4-trifluoromethoxy-benzyl)-amino]-propyl}-1,2,3,4-tetrahydro-isoquinolin-7-yl)-acetic acid

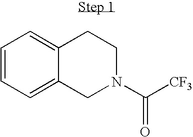

1-(3,4-Dihydro-1H-isoquinolin-2-yl)-2,2,2-trifluoro-ethanone: To cold trifluoroacetic anhydride (98.1 g, 467 mmol) was slowly added 1,2,3,4-tetrahydroisoquinoline (29.6 mL, 234 mmol). After addition, the cooling bath was removed and the reaction mixture was stirred at room temperature for 20 h. The reaction mixture was concentrated under reduced pressure and the residue was purified by distillation to give 50.17 g (94%) of the desired product as a colorless oil. ¹H NMR (400 MHz, CDCl₃) δ 7.22 (m, 4H), 4.77 (d, 2H), 3.85 (m, 2H), 2.96 (m, 2H).

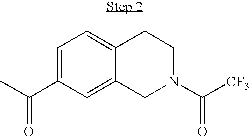

1-(7-Acetyl-3,4-dihydro-1H-isoquinolin-2-yl)-2,2,2-trifluoro-ethanone: To a suspension of 1-(3,4-dihydro-1H-isoquinolin-2-yl)-2,2,2-trifluoro-ethanone (50.17 g, 219 mmol) and AlCl₃ (175 g, 1.31 mol) in CS₂ (300 mL) was added acetyl chloride (47 mL, 657 mmol) at a rate that kept gentle refluxing. After addition, the mixture was heated at reflux for 1 h and then stirred at room temperature overnight. The reaction mixture was concentrated under reduced pressure and the residue was carefully added to ice-cooled 3N HC (1000 mL). The resulting mixture was extracted with ethyl acetate (500 mL×3). The combined organic layer was washed with water, saturated NaHCO₃, brine and then dried over Na₂SO₄. After removal of solvent, the residue was triturated with hexanes and the solid was recrystallized from MeOH—H₂O to give 36.05 g (61%) of the desired product as a white solid. 1 NMR (400 MHz, CDCl₃) δ 7.81 (t, 1H), 7.74 (d, 1H), 7.26 (dd, 1H), 4.82 (d, 2H), 3.88 (m, 2H), 3.02 (m, 2H), 2.59 (s, 3H).

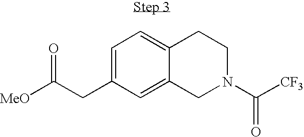

[2-(2,2,2-Trifluoro-acetyl)-1,2,3,4-tetrahydro-isoquinolin-7-yl]-acetic acid methyl ester: To a solution of 1-(7-acetyl-3,4-dihydro-1H-isoquinolin-2-yl)-2,2,2-trifluoro-ethanone (4.21 g, 15.5 mmol) and 70% HClO₄ (5.6 mL, 93.1 mmol) in MeOH (30 mL) was added T1(NO₃)₃ (10.35 g, 23.3 mmol) at room temperature with stirring. The mixture was stirred at room temperature for 4 h and then was filtered. The filtrate was concentrated under reduced pressure and the residue was taken up with water (100 mL) and extracted with CH₂Cl₂ (50 mL×3). The combined organic layers were washed with water, brine and then dried over Na₂SO₄. After removal of solvent, the residue was purified by silica gel chromatography to give 2.11 g (45%) of the desired product. ¹H NMR (400 MHz, CDCl₃) δ 7.13 (d, 1H), 7.12 (s, 1H), 7.05 (d, 1H), 4.75 (d, 2H), 3.87 (t, 1H), 3.83 (t, 1H), 3.70 (s, 3H), 3.60 (s, 2H), 2.94 (m, 2H).

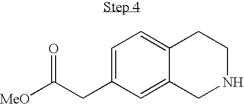

(1,2,3,4-Tetrahydro-isoquinolin-7-yl)-acetic acid methyl ester: To a solution of [2-(2,2,2-trifluoro-acetyl)-1,2,3,4-tetrahydro-isoquinolin-7-yl]-acetic acid methyl ester (2.11 g, 7.0 mmol) in MeOH (50 mL) was added NaBH₄ (530 mg, 14.0 mmol) in three portions at 0° C. with stirring. After addition, the reaction mixture was stirred for 15 min and then the cooling bath was removed. After stirring at room temperature for 5 h, the reaction mixture was concentrated under reduced pressure and the residue was taken up with water (100 mL) and extracted with ethyl acetate (100 mL×2). The combined organic layers were washed with water, brine and then dried over Na₂SO₄. After removal of solvent, the residue was purified by silica gel chromatography to give 1.30 g (91%) of the desired product as a colorless oil. ¹H NMR (400 MHz, CDCl₃) δ 7.04 (s, 2H), 6.92 (s, 1H), 3.99 (s, 2H), 3.68 (s, 3H), 3.56 (s, 2H), 3.12 (t, 2H), 2.77 (t, 2H).

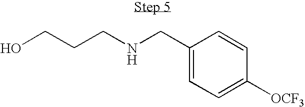

3-(4-Trifluoromethoxy-benzylamino)-propan-1-ol: To a solution of 4-trifluoro-methoxybenzaldehyde (15.0 g, 78.9 mmol) and 3-aminopropan-1-ol (6.6 mL, 86.8 mmol) in 3:1 of MeOH/trimethylorthoformate (150 mL) was added NaBH₄ (4.48 g, 118.4 mmol) in three portions at 0° C. with stirring. After addition, the reaction mixture was stirred for 15 min and then the cooling bath was removed. After stirring at room temperature for 5 h, the reaction mixture was concentrated under reduced pressure and the residue was taken up with water (250 mL) and extracted with ethyl acetate (250 mL×2). The combined organic layers were washed with water, brine and then dried over Na₂SO₄. Removal of solvent gave 19.05 g (97%) of the desired product as a colorless oil, which was used in the next step without purification. ¹H NMR (400 MHz, CDCl₃) δ 7.32 (d, 2H), 7.16 (d, 2H), 3.81 (t, 2H), 3.78 (s, 2H), 2.89 (t, 2H), 1.73 (m, 2H).

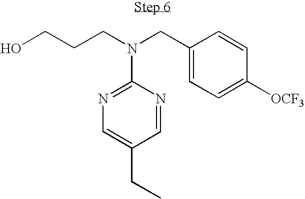

3-[(5-Ethyl-pyrimidin-2-yl)-(4-trifluoromethoxy-benzyl)-amino]-propan-1-ol: A solution of 3-(4-trifluoromethoxy-benzylamino)-propan-1-ol (3.1 g, 12.4 mmol), 2-chloro-5-ethylpyrimidine (1.51 mL, 12.4 mmol) and K₂CO₃ (2.6 g, 18.7 mmol) in DMF (50 mL) was heated to 165° C. overnight in a sealed tube. After cooling to room temperature, the mixture was diluted with ethyl acetate (100 mL) and then washed with water, brine and dried over Na₂SO₄. After removal of solvent, the residue was purified by silica gel chromatography to give 2.15 g (49%) of the desired product as a colorless solid. ¹H NMR (400 MHz, CDCl₃) δ 8.18 (s, 2H), 7.26 (d, 2H), 7.13 (d, 2H), 4.83 (s, 2H), 3.71 (t, 2H), 3.52 (t, 2H), 2.48 (q, 2H), 1.73 (m, 2H), 1.20 (t, 3H).

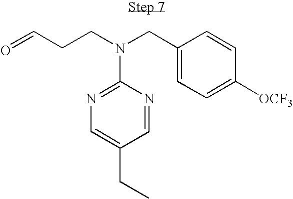

3-[(5-Ethyl-pyrimidin-2-yl)-(4-trifluoromethoxy-benzyl)-amino]-propionaldehyde: To a solution of 3-[(5-ethyl-pyrimidin-2-yl)-(4-trifluoromethoxy-benzyl)-amino]-propan-1-ol (2.05 g, 5.8 mmol) in dichloromethane (20 mL) was added Dess-Martin reagent (2.9 g, 6.9 mmol). The resulting mixture was stirred at room temperature under N₂ for 4 h. A 1:1 saturated Na₂S₂SO₃/saturated NaHCO₃ solution (10 mL) was added to the reaction mixture. After separation, the aqueous solution was extracted with CH₂Cl₂ (50 mL). The organic solution was washed with water, brine and then dried over Na₂SO₄. Removal of solvent gave 1.4 g of the desired product which was used without purification.

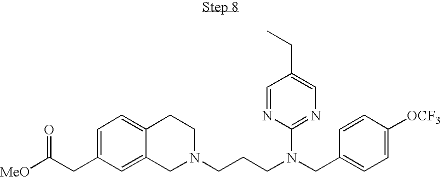

(2-{3-[(5-Ethyl-pyrimidin-2-yl)-(4-trifluoromethoxy-benzyl)-amino]-propyl}-1,2,3,4-tetrahydro-isoquinolin-7-yl)-acetic acid methyl ester: To a solution of 3-[(5-ethyl-pyrimidin-2-yl)-(4-trifluoromethoxy-benzyl)-amino]-propionaldehyde (1.4 g, 4.0 mmol) and (1,2,3,4-tetrahydro-isoquinolin-7-yl)-acetic acid methyl ester (813 mg, 4.0 mmol) in 3:1 of MeOH/trimethylorthoformate (40 mL) was added NaBH₄ (225 mg, 5.9 mmol) in three portions at 0° C. with stirring. After addition, the reaction mixture was stirred for 15 min and then the cooling bath was removed. After stirring at room temperature for 5 h, the reaction mixture was concentrated under reduced pressure and the residue was taken up with water (250 mL) and extracted with ethyl acetate (50 mL×2). The combined organic layers were washed with water, brine and then dried over Na₂SO₄. After removal of solvent, the residue was purified by silica gel chromatography to give 125 mg (6% yield) of product as a colorless oil. ¹H NMR (400 MHz, CDCl₃) δ 8.18 (s, 2H), 7.24 (d, 2H), 7.11 (d, 2H), 7.04 (s, 2H), 6.92 (s, 1H), 4.89 (s, 2H), 3.67 (s, 3H), 3.64 (t, 2H), 3.57 (s, 2H), 3.56 (s, 2H), 2.85 (t, 2H), 2.67 (t, 2H), 2.50 (m, 4H), 1.90 (m, 2H), 1.20 (t, 3H).

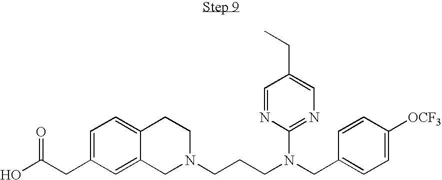

(2-{3-[(5-Ethyl-pyrimidin-2-yl)-(4-trifluoromethoxy-benzyl)-amino]-propyl}-1,2,3,4-tetrahydro-isoquinolin-7-yl)-acetic acid: A solution of (2-{3-[(5-ethyl-pyrimidin-2-yl)-(4-trifluoromethoxy-benzyl)-amino]-propyl}-1,2,3,4-tetrahydro-isoquinolin-7-yl)-acetic acid methyl ester (124.5 mg, 0.23 mmol) and 1N LiOH (0.92 mL, 0.92 mmol) in 3:1 THF/MeOH (10 mL) was stirred at room temperature for 3 h. The reaction mixture was concentrated under reduced pressure and the residue was taken up with water (5 mL) and neutralized with 1N HCl (0.92 mL). The resulting mixture was extracted with ethyl acetate (10 mL). The organic layer was washed with water, brine and then dried over Na₂SO₄. Removal of solvent gave the title compound 80 mg (66%). ¹H NMR (400 MHz, CDCl₃) δ 8.18 (s, 2H), 7.24 (d, 2H), 7.11 (d, 2H), 7.03 (d, 1H), 6.93 (d, 1H), 6.83 (s, 1H), 4.85 (s, 2H), 3.79 (s, 2H), 3.62 (t, 2H), 3.39 (s, 2H), 2.96 (m, 2H), 2.89 (m, 2H), 2.78 (t, 2H), 2.47 (q, 2H), 2.00 (m, 2H), 1.20 (t, 3H).

### EXAMPLE 2

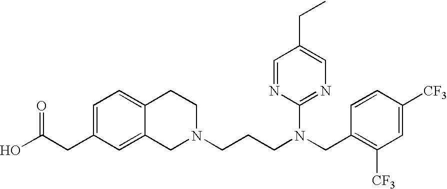

(2-{3-[(2,4-Bis-trifluoromethyl-benzyl)-(5-ethyl-pyrimidin-2-yl)-amino]-propyl}-1,2,3,4-tetrahydro-isoquinolin-7-yl)-acetic acid: The title compound was prepared as outlined in Example 1 using 2,4-bis(trifluoromethyl)benzaldehyde. ¹H NMR (400 MHz, CDCl₃) δ 8.18 (s, 2H), 7.91 (d, 1H), 7.67 (d, 1H), 7.34 (d, 1H), 7.05 (d, 1H), 6.98 (d, 1H), 6.87 (s, 1H), 5.09 (s, 2H), 3.95 (s, 2H), 3.68 (t, 2H), 3.42 (s, 2H), 3.13 (m, 2H), 2.97 (m, 2H), 2.92 (t, 2H), 2.47 (q, 2H), 2.13 (m, 2H), 1.20 (t, 3H).

### EXAMPLE 3

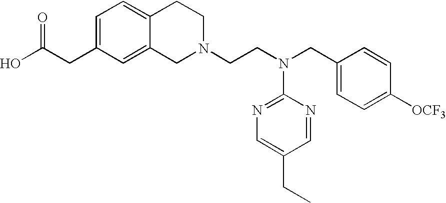

(2-{2-[(5-Ethyl-pyrimidin-2-yl)-(4-trifluoromethoxy-benzyl)-amino]-ethyl}-1,2,3,4-tetrahydro-isoquinolin-7-yl)-acetic acid: The title compound was prepared as outlined in Example 1 using ethanolamine. ¹H NMR (400 MHz, CDCl₃) δ 8.17 (s, 2H), 7.27 (d, 2H), 7.11 (d, 2H), 7.01 (d, 1H), 6.93 (d, 1H), 6.83 (s, 1H), 4.88 (s, 2H), 3.86 (t, 2H), 3.76 (s, 2H), 3.39 (s, 2H), 2.94 (m, 2H), 2.88 (m, 4H), 2.46 (q, 2H), 1.19 (t, 3H).

### EXAMPLE 4

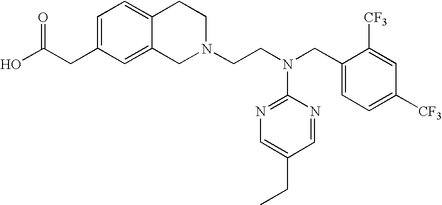

(2-{2-[(2,4-Bis-trifluoromethyl-benzyl)-(5-ethyl-pyrimidin-2-yl)-amino]-ethyl}-1,2,3,4-tetrahydro-isoquinolin-7-yl)-acetic acid: The title compound was prepared as outlined in Example 3 using 2,4-bis(trifluoromethyl)benzaldehyde. ¹H NMR (400 MHz, CDCl₃) δ 8.17 (s, 2H), 7.90 (s, 1H), 7.65 (d, 1H), 7.37 (d, 1H), 6.99 (d, 1H), 6.93 (d, 1H), 6.81 (s, 1H), 5.12 (s, 2H), 3.93 (t, 2H), 3.77 (d, 2H), 3.38 (s, 2H), 2.94 (m, 4H), 2.87 (m, 2H), 2.47 (q, 2H), 1.19 (t, 3H).

### EXAMPLE 5

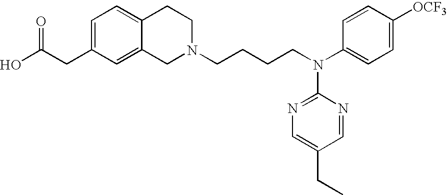

### (2-{4-[(5-Ethyl-pyrimidin-2-yl)-(4-trifluoromethoxy-phenyl)-amino]-butyl}-1,2,3,4-tetrahydro-isoquinolin-7-yl)-acetic acid

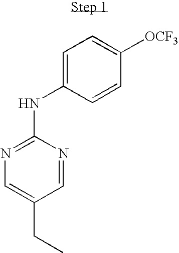

2 (5-Ethyl-pyrimidin-2-yl)-(4-trifluoromethoxy-phenyl)-amine: To a solution of 4-trifluoromethoxyphenylamine (0.5 mL, 3.72 mmol) and 2-chloro-5-ethyl-pyrimidine (0.45 mL, 3.72 mmol) in toluene (5 mL) was added Pd(AcO)₂ (251 mg, 0.37 mmol), rac-BINAP (347 mg, 0.56 mmol) and Cs₂CO₃ (1.8 g, 5.6 mmol). The resulting mixture was heated in a microwave oven at 150° C. for 30 min. After cooling to room temperature, the reaction mixture was purified by silica gel chromatography to give the desired product, 252 mg (24%) as a yellow solid. ¹H NMR (400 MHz, CDCl₃) δ 8.29 (s, 2H), 7.63 (d, 2H), 7.17 (d, 2H), 2.54 (q, 2H), 1.24 (t, 3H).

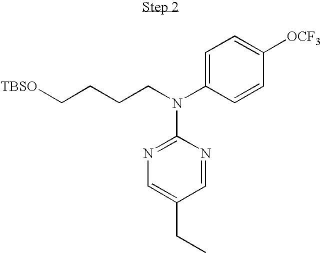

[[4-(tert-Butyl-dimethyl-silanyloxy)-butyl]-(5-ethyl-pyrimidin-2-yl)-(4-trifluoromethyl-phenyl)]amine: To a solution of 2 (5-ethyl-pyrimidin-2-yl)-(4-trifluoromethoxy-phenyl)amine (252 mg, 0.89 mmol) and (4-bromo-butoxy)-tert-butyl-dimethylsilane (285 mg, 1.07 mmol) in THF (10 mL) was added NaH (60% in mineral oil) (54 mg, 1.34 mmol). The resulting mixture was heated to 65° C. under N₂ with stirring. After heating for 16 h, the reaction mixture was cooled to room temperature, quenched with water (10 mL) and extracted with ethyl acetate (25 mL×2). The combined organic layers were washed with water, brine and then dried over Na₂SO₄. After removal of solvent, the crude product was purified by silica gel chromatography to give 100 mg (24%) of the desired product. ¹H NMR (400 MHz, CDCl₃) δ 8.17 (s, 2H), 7.29 (d, 2H), 7.22 (d, 2H), 3.98 (t, 2H), 3.61 (t, 2H), 2.47 (q, 2H), 1.68 (m, 2H), 1.55 (m, 2H), 1.15 (t, 3H), 0.85 (s, 9H), 0.01 (s, 6H).

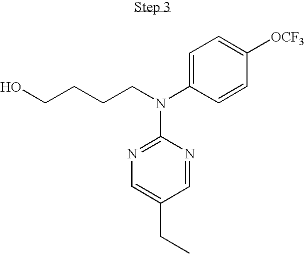

4-[(5-Ethyl-pyrimidin-2-yl)-(4-trifluoromethoxy-phenyl)-amino]-butan-1-ol: To a solution of [[4-(tert-butyl-dimethyl-silanyloxy)-butyl]-(5-ethyl-pyrimidin-2-yl)-(4-trifluoromethyl-phenyl)] amine (657 mg, 1.4 mmol) in THF (10 mL) was added TBAF (1N solution in THF) (2.8 mL, 2.8 mmol). After stirring at room temperature for 2 h, the reaction mixture was diluted with ethyl acetate (50 mL) and then washed with water, brine and dried over Na₂SO₄. After removal of solvent, the crude product was purified by silica gel chromatography to give 498 mg (99%) of the desired product. ¹H NMR (400 MHz, CDCl₃) δ 8.18 (s, 2H), 7.29 (d, 2H), 7.23 (d, 2H), 3.99 (t, 2H), 3.72 (t, 2H), 2.47 (q, 2H), 1.77 (m, 2H), 1.61 (m, 2H), 1.12 (t, 3H).

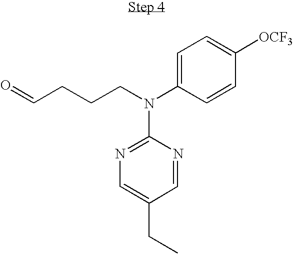

4-[(5-Ethyl-pyrimidin-2-yl)-(4-trifluoromethoxy-phenyl)-amino]-butyraldehyde: To a solution of 4-[(5-ethyl-pyrimidin-2-yl)-(4-trifluoromethoxy-phenyl)-amino]-butan-1-ol (803 mg, 2.26 mmol) in CH₂Cl₂ (10 mL) was added Dess-Martin reagent (1.9 g, 4.52 mmol). After stirring at room temperature for 2 h, the reaction was quenched with 1:1 saturated NaHCO₃/saturated Na₂S₂O₃ solution (10 mL). After separation, the aqueous layer was extracted with CH₂Cl₂ (50 mL). The organic solution was washed with water, brine and dried over Na₂SO₄. After removal of solvent, the crude product was used in next step without purification.

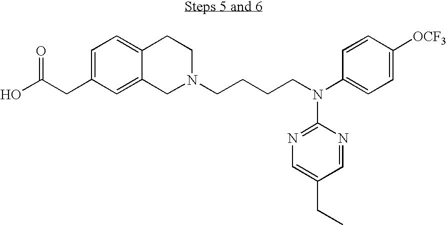

(2-{4-[(5-Ethyl-pyrimidin-2-yl)-(4-trifluoromethoxy-phenyl)-amino]-butyl}-1,2,3,4-tetrahydro-isoquinolin-7-yl)-acetic acid: The title compound was prepared as outlined in Example 1 using the compound from the previous step. ¹H NMR (400 MHz, CDCl₃) δ 8.18 (s, 2H), 7.39 (d, 2H), 7.33 (d, 2H), 7.18 (d, 1H), 7.11 (d, 1H), 7.06 (s, 1H), 4.07 (t, 2H), 3.67 (s, 2H), 3.38 (s, 2H), 3.15 (m, 2H), 3.06 (m, 2H), 2.51 (m, 2H), 2.50 (q, 2H), 1.82 (m, 2H), 1.75 (m, 2H), 1.19 (t, 3H).

### EXAMPLE 6

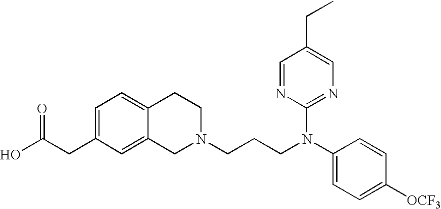

(2-{3-[(5-Ethyl-pyrimidin-2-yl)-(4-trifluoromethoxy-phenyl)-amino]-propyl}-1,2,3,4-tetrahydroisoquinolin-7-yl)-acetic acid: The title compound was prepared as outlined in Example 5 using (3-bromo-propoxy)-tert-butyl-dimethylsilane. ¹H NMR (400 MHz, CDCl₃) δ 8.18 (s, 2H), 7.28 (d, 2H), 7.21 (d, 2H), 7.00 (d, 1H), 6.92 (d, 1H), 6.81 (s, 1H), 4.03 (t, 2H), 3.76 (s, 2H), 3.36 (s, 2H), 2.92 (m, 2H), 2.87 (m, 2H), 2.78 (t, 2H), 2.47 (q, 2H), 2.05 (m, 2H), 1.21 (t, 3H).

### EXAMPLE 7

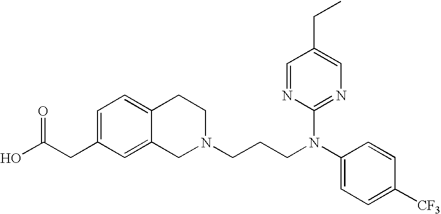

(2-[3-[(5-Ethyl-pyrimidin-2-yl)-(4-trifluoromethyl-phenyl)-amino]-propyl]-1,2,3,4-tetrahydroisoquinolin-7-yl)-acetic acid: The title compound was prepared as outlined in Example 6 using 4-trifluoromethylphenylamine. ¹H NMR (400 MHz, CDCl₃) δ 8.20 (s, 2H), 7.61 (d, 2H), 7.39 (d, 2H), 7.02 (d, 1H), 6.95 (d, 1H), 6.82 (s, 1H), 4.09 (t, 2H), 3.87 (s, 2H), 3.38 (s, 2H), 3.05 (m, 2H), 2.91 (m, 4H), 2.49 (q, 2H), 2.11 (m, 2H), 1.20 (t, 3H).

### EXAMPLE 8

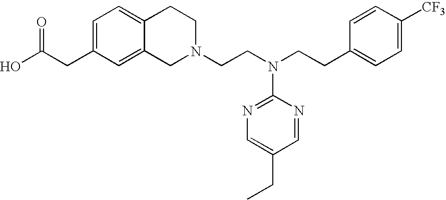

### [2-(2-{(5-Ethyl-pyrimidin-2-yl)-[2-(4-trifluoromethyl-phenyl)-ethyl]-amino}-ethyl)-1,2,3,4-tetrahydro-isoquinolin-7-yl]-acetic acid

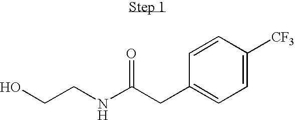

N-(2-Hydroxy-ethyl)-2-(4-trifluoromethyl-phenyl)-acetamide: To suspension of (4-trifluoromethyl-phenyl) acetic acid (5.2 g, 25.5 mmol) and ethanolamine (1.84 mL, 30.6 mmol) in acetonitrile (100 mL) was added 1-[3-(dimethylamino)propyl]-3-ethylcarbodiimide hydrochloride (5.9 g, 30.6 mmol), 1-hydroxybenzotriazole (3.4 g, 25.5 mmol) and triethylamine (7.1 mL, 50.9 mmol). The resulting mixture was stirred at room temperature for 6 h. The reaction mixture was concentrated under reduced pressure and water (200 mL) was added to the residue. The aqueous mixture was extracted with ethyl acetate (200 mL×2). The combined organic extracts were washed with water, brine and dried over Na₂SO₄. The organic solution was concentrated under reduced pressure to give 3.22 g (51%) of the desired product which was used in next step without purification. ¹H NMR (400 MHz, CDCl₃) δ 7.60 (d, 2H), 7.40 (d, 2H), 5.99 (b, 1H), 3.69 (t, 2H), 3.62 (s, 2H), 3.41 (t, 2H).

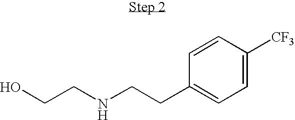

2-[2-(4-Trifluoromethyl-phenyl)-ethylamino]-ethanol: To a 2M solution of LiBH₄ in THF (28.7 mL, 57.3 mmol) was added trimethylsilyl chloride (14.5 mL, 114.6 mmol) at room temperature with stirring. After 20 min a white precipitate was formed. To the resulting suspension was added a solution of N-(2-hydroxy-ethyl)-2-(4-trifluoromethyl-phenyl)-acetamide (3.22 g, 13.0 mmol) in THF (30 mL). The reaction mixture was stirred at room temperature for 20 h. The reaction was quenched by slow addition of MeOH (10 mL). The mixture was concentrated under reduced pressure and the residue was taken up in 20% KOH (25 mL) and water (100 mL). The aqueous mixture was extracted with ethyl acetate (100 mL×2). The combined organic layer was washed with water, brine and dried over Na₂SO₄. The organic solution was concentrated under reduced pressure to give 2.8 g (93%) of the desired product which was used in next step without purification. ¹H NMR (400 MHz, CDCl₃) δ 7.55 (d, 2H), 7.31 (d, 2H), 3.63 (t, 2H), 2.92 (t, 2H), 2.86 (t, 2H), 2.79 (t, 2H).

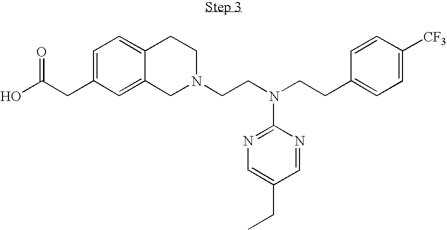

[2-(2-{(5-Ethyl-pyrimidin-2-yl)-[2-(4-trifluoromethyl-phenyl)-ethyl]-amino}-ethyl)-1,2,3,4-tetrahydro-isoquinolin-7-yl]-acetic acid: The title compound was prepared as outlined in Example 1 using the compound from the previous step. ¹H NMR (400 MHz, CDCl₃) δ 8.16 (s, 2H), 7.50 (d, 2H), 7.35 (d, 2H), 7.07 (d, 1H), 6.99 (d, 1H), 6.89 (s, 1H), 3.99 (b, 2H), 3.92 (s, 2H), 3.83 (t, 2H), 3.46 (s, 2H), 3.16 (b, 2H), 2.98 (m, 6H), 2.46 (q, 2H), 1.19 (t, 3H).

### EXAMPLE 9

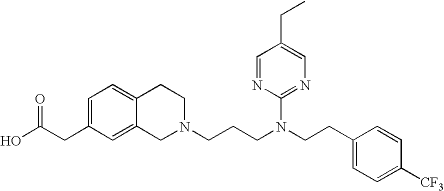

[2-(3-{(5-Ethyl-pyrimidin-2-yl)-[2-(4-trifluoromethyl-phenyl)-ethyl]-amino}-propyl)-1,2,3,4-tetrahydro-isoquinolin-7-yl]-acetic acid: The title compound was prepared as outlined in Example 8 using 3-aminopropan-1-ol. ¹H NMR (400 MHz, CDCl₃) δ 8.17 (s, 2H), 7.51 (d, 2H), 7.34 (d, 2H), 7.01 (d, 1H), 6.90 (d, 1H), 6.81 (s, 1H), 3.77 (m, 4H), 3.54 (t, 2H), 3.36 (s, 2H), 2.94 (m, 6H), 2.76 (t, 2H), 2.46 (q, 2H), 2.01 (m, 2H), 1.20 (t, 3H).

### EXAMPLE 10

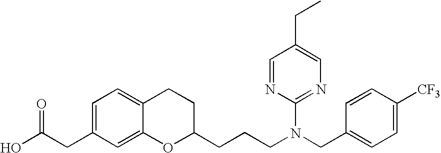

### (2-{3-[(5-Ethyl-pyrimidin-2-yl)-(4-trifluoromethyl-benzyl)-amino]-propyl}-chroman-7-yl)-acetic acid

### EXAMPLE 11

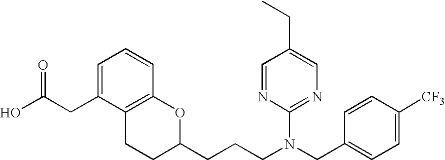

### (2-{3-[(5-Ethyl-pyrimidin-2-yl)-(4-trifluoromethyl-benzyl)-amino]-propyl}-chroman-5-yl)-acetic acid

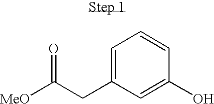

3-Hydroxyphenylacetic acid methyl ester: To a solution of 3-hydroxyphenylacetic acid (50.66 g, 333 mmol) in methanol (200 mL) was added a drop of concentrated HCl. The resulting solution was heated to reflux for 6 h, cooled, and concentrated under reduced pressure to give 55.94 g (99% yield) of the desired product which was used in the next step without purification. ¹H NMR (400 MHz, CDCl₃) δ 7.19 (t, 1H), 6.91 (d, 1H), 6.89 (s, 1H), 6.87 (d, 1H), 3.70 (s, 3H), 3.58 (s, 2H).

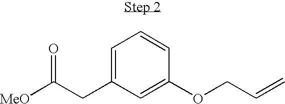

3-Allyloxyphenylacetic acid methyl ester: A mixture of 3-hydroxyphenylacetic acid methyl ester (34.25 g, 206 mmol), allylbromide (17.84 mL, 206 mmol) and K₂CO₃ (28.5 g, 206 mmol) in acetone (500 mL) was heated to reflux with vigorous stirring. After 24 h, the reaction mixture was cooled and concentrated under reduced pressure. The residue was taken up in 20% KOH (100 mL), diluted with water (200 mL) and then extracted with ether (500 mL×3). The combined organic layer was washed with brine, dried over Na₂SO₄ and concentrated under reduced pressure to give 35.64 g of the desired product which was used in the next step without purification. ¹H NMR (400 MHz, CDCl₃) δ 7.24 (d, 1H), 6.85 (m, 3H), 6.05 (m, 1H), 5.41 (d, 1H), 5.28 (d, 1H), 4.54 (m, 2H), 3.69 (s, 3H), 3.60 (2H).

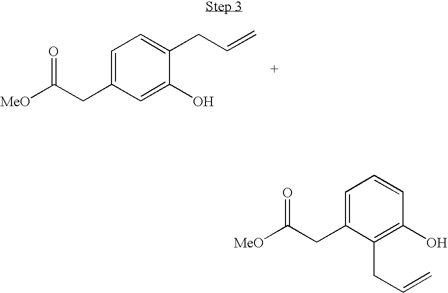

(4-Allyl-3-hydroxy-phenyl)-acetic acid methyl ester and (2-Allyl-3-hydroxy-phenyl)-acetic acid methyl ester: 3-Allyloxyphenylacetic acid methyl ester (35.64 g, 172.8 mmol) was sealed in a high pressure reaction tube and heated to 220° C. in a microwave oven for 3 h. After cooling, a mixture of the above regioisomers (˜1:1.28 by ¹H NMR) was obtained which were used in the next step without purification. ¹H NMR (400 MHz, CDCl₃) δ 7.07 (m, 2H), 6.84-6.74 (m, 4H), 5.98 (m, 2H), 5.08 (m, 4H), 3.69 (s, 3H), 3.68 (s, 3H), 3.64 (s, 2H), 3.56 (s, 2H), 3.46 (dt, 2H), 3.38 (d, 2H).

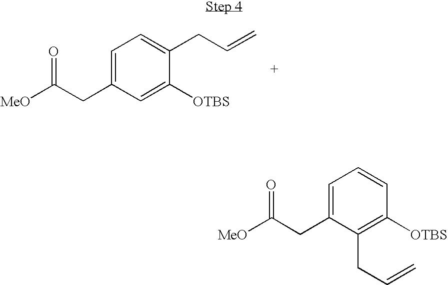

[4-Allyl-3-(tert-butyl-dimethyl-silanyloxy)-phenyl]-acetic acid methyl ester and [2-Allyl-3-(tert-butyl-dimethyl-silanyloxy)-phenyl]-acetic acid methyl ester: To a solution of (4-allyl-3-hydroxy-phenyl)-acetic acid methyl ester and (2-allyl-3-hydroxy-phenyl)-acetic acid methyl ester (33 g, 160 mmol) in DMF (75 mL) was added TBSC (28.9 g, 192 mmol) and imidazole (21.8 g, 320 mmol). The resulting mixture was stirred for 4 h at room temperature. The mixture was diluted with water (250 mL) and extracted with ethyl acetate (250 mL×2). The combined organic layer was washed with water, brine, dried over Na₂SO₄ and concentrated under reduced pressure to give 51 g (99%) of the desired compounds which were used in the next step without purification. ¹H NMR (400 MHz, CDCl₃) δ 7.06 (m, 2H), 6.79 (m, 4H), 5.90 (m, 2H), 4.97 (m, 4H), 3.68 (s, 3H), 3.67 (s, 3H), 3.62 (s, 2H), 3.54 (s, 2H), 3.43 (dt, 2H), 3.34 (d, 2H), 1.01 (s, 9H), 1.00 (s, 9H), 0.23 (s, 3H), 0.22 (s, 3H).

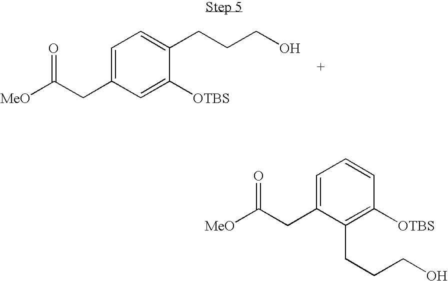

[3-(tert-Butyl-dimethyl-silanyloxy)-4-(3-hydroxy-propyl)-phenyl]-acetic acid methyl ester and [3-(tert-Butyl-dimethyl-silanyloxy)-2-(3-hydroxy-propyl)-phenyl]-acetic acid methyl ester: A solution of [4-allyl-3-(tert-butyl-dimethyl-silanyloxy)-phenyl]-acetic acid methyl ester and [2-allyl-3-(tert-butyl-dimethyl-silanyloxy)-phenyl]-acetic acid methyl ester (5.47 g, 17.07 mmol) in THF (70 mL) was cooled to 0° C. To the cold solution was slowly added BH₃.THF (1M in THF) (18.77 mL, 18.77 mmol). The reaction mixture was stirred for 20 min and then at room temperature for 3 h. The mixture was cooled to 0° C. To the cold mixture was added 30% H₂O₂ (7 mL) followed by 3M NaOH (7 mL). The reaction mixture was stirred for 10 min and then at room temperature for 3 h. The mixture was concentrated under reduced pressure, dissolved in 1M KOH (100 mL) and extracted with ethyl acetate (100 mL×2). The combined organic layer was washed with water, brine, dried over Na₂SO₄ and concentrated under reduced pressure. The residue was purified by chromatography to give 3.0 g (52%) of the above mixture. ¹H NMR (400 MHz, CDCl₃) δ 7.07 (t, 1H), 7.02 (d, 1H), 6.84 (d, 1H), 6.79 (dd, 1H), 6.74 (m, 2H), 3.69 (s, 6H), 3.68 (s, 2H), 3.62 (t, 4H), 3.54 (s, 2H), 2.75 (t, 2H), 2.66 (t, 2H), 1.82 (m, 2H), 1.76 (m, 2H), 1.56 (b, 2H), 1.01 (s, 18H), 0.24 (s, 6H).

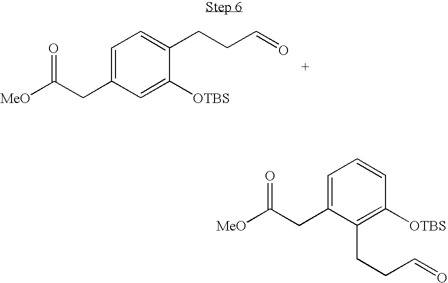

[3-(tert-Butyl-dimethyl-silanyloxy)-4-(3-oxo-propyl)-phenyl]-acetic acid methyl ester and [3-(tert-Butyl-dimethyl-silanyloxy)-2-(3-oxo-propyl)-phenyl]-acetic acid methyl ester: To a solution of [3-(tert-butyl-dimethyl-silanyloxy)-4-(3-hydroxy-propyl)-phenyl]-acetic acid methyl ester and [3-(tert-butyl-dimethyl-silanyloxy)-2-(3-hydroxy-propyl)-phenyl]-acetic acid methyl ester (3.0 g, 9.42 mmol) in CH₂Cl₂ (30 mL) was added Dess-Martin solution (15 wt % in CH₂Cl₂) (32 mL, 11.30 mmol). The resulting mixture was stirred at room temperature for 3 h. To the reaction mixture was added saturated aqueous Na₂S₂O₃ (15 mL) followed by saturated aqueous NaHCO₃ (15 mL). The aqueous layer was extracted with CH₂Cl₂ (100 mL) and the organic layer was washed with water, brine, dried over Na₂SO₄ and concentrated under reduced pressure to give 3.0 g (99%) of the desired products which were used in the next step without purification. ¹H NMR (400 MHz, CDCl₃) δ 9.81 (m, 2H), 7.06 (m, 2H), 6.82 (d, 1H), 6.78 (d, 1H), 6.74 (m, 2H), 3.68 (s, 3H), 3.67 (s, 3H), 3.66 (s, 2H), 3.54 (s, 2H), 2.91 (m, 4H), 2.70 (m, 4H), 1.01 (s, 9H), 0.99 (s, 9H), 0.25 (s, 12H).

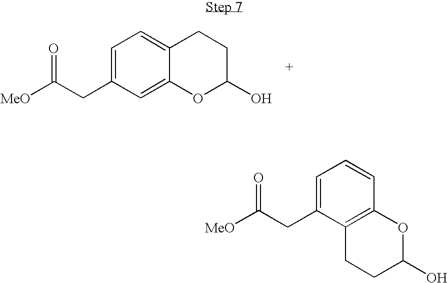

(2-Hydroxy-chroman-7-yl)-acetic acid methyl ester and (2-Hydroxy-chroman-5-yl)-acetic acid methyl ester: To a solution of [3-(tert-butyl-dimethyl-silanyloxy)-4-(3-oxo-propyl)-phenyl]-acetic acid methyl ester and [3-(tert-butyl-dimethyl-silanyloxy)-2-(3-oxo-propyl)-phenyl]-acetic acid methyl ester (3.0 g, 9.42 mmol) in THF (30 mL) was added TBAF (1M in THF) (11.30 mL, 11.3 mmol). After stirring 1 h, the solution was concentrated under reduced pressure. The residue was diluted with ethyl acetate (100 mL), washed with water, brine and dried over Na₂SO₄. The solution was concentrated under reduced pressure and the residue was purified by chromatography to give 1.5 g (72%) of the desired products. ¹H NMR (400 MHz, CDCl₃) δ 7.09 (t, 1H), 7.01 (d, 1H), 6.80 (m, 4H), 5.58 (m, 2H), 3.69 (s, 3H), 3.68 (s, 3H), 3.61 (s, 2H), 3.55 (s, 2H), 2.95 (m, 1H), 2.85 (m, 1H), 2.70 (m, 2H), 2.43 (b, 1H), 2.02 (m, 4H), 1.42 (b, 1H).

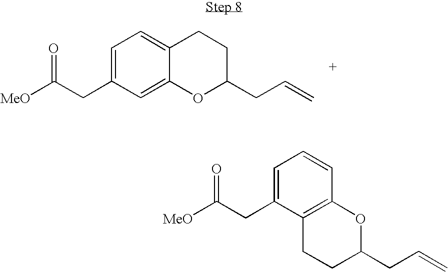

(2-Allyl-chroman-7-yl)-acetic acid methyl ester and (2-Allyl-chroman-5-yl)-acetic acid methyl ester: To a solution of (2-hydroxy-chroman-7-yl)-acetic acid methyl ester and (2-hydroxy-chroman-5-yl)-acetic acid methyl ester (5.03 g, 22.63 mmol) in CH₂Cl₂ (110 mL) at 0° C. was added allyltrimethylsilane (7.3 mL, 45.27 mmol). To the cold solution was slowly added BF₃.OEt₂ (5.7 mL, 45.27 mmol) and the solution was kept at 20 min. The mixture was warmed to room temperature and stirred for 3 h. The mixture was cooled to 0° C. and saturated aqueous NaHCO₃ (100 mL) was slowly added to the mixture. After 15 min, saturated NaHCO₃ was added until PH˜7. The organic layer was separated and aqueous layer was extracted with CH₂Cl₂ (150 mL×2). The combined organic layer was washed with water, brine and dried over Na₂SO₄. The solution was concentrated under reduced pressure and the residue was purified by chromatography to give 2.3 g (41%) of the desired compounds. ¹H NMR (400 MHz, CDCl₃) δ 7.06 (t, 1H), 6.98 (d, 1H), 6.76 (m, 4H), 5.91 (m, 2H), 5.13 (m, 4H), 4.01 (m, 2H), 3.69 (s, 3H), 3.68 (s, 3H), 3.59 (s, 2H), 3.53 (s, 2H), 2.72 (m, 4H), 2.52 (m, 2H), 2.40 (m, 2H), 2.04 (m, 2H), 1.73 (m, 2H).

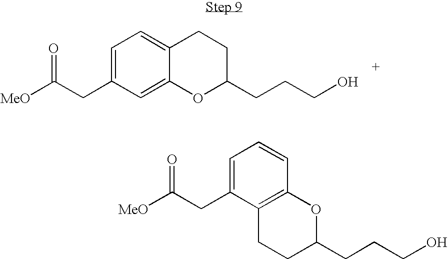

[2-(3-Hydroxy-propyl)-chroman-7-yl]-acetic acid methyl ester and [2-(3-Hydroxy-propyl)-chroman-5-yl]-acetic acid methyl ester: To a solution of (2-allyl-chroman-7-yl)-acetic acid methyl ester and (2-allyl-chroman-5-yl)-acetic acid methyl ester (415 mg, 1.68 mmol) in THF (20 mL) at 0° C. was slowly added BH₃. THF (1M in THF) (1.85 mL, 1.85 mmol). After 20 min, the cooling bath was removed and the mixture was stirred at room temperature for 2 h. The mixture was cooled to 0° C. and 30% H₂O₂ (1 mL) was added followed by 3M NaOH (1 mL). After 10 min, the cooling bath was removed and the mixture was stirred at room temperature for 1 h. The reaction mixture was diluted with ethyl acetate (50 mL), washed with water, brine and dried over Na₂SO₄. The solution was concentrated under reduced pressure and the residue was purified by chromatography to give 365 mg (80%) of the above mixture. ¹H NMR (400 MHz, CDCl₃) δ 7.06 (t, 1H), 6.98 (d, 1H), 6.75 (m, 4H), 3.98 (m, 2H), 3.73 (b, 4H), 3.69 (s, 3H), 3.68 (s, 3H), 3.58 (s, 2H), 3.53 (s, 2H), 2.80 (m, 2H), 2.73 (m, 4H), 2.02 (m, 2H), 1.76 (m, 8H).

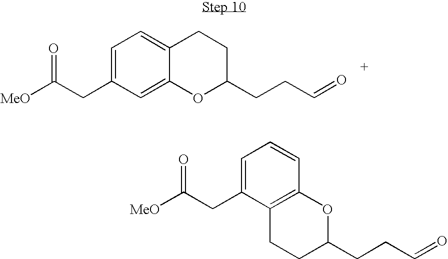

[2-(3-Oxo-propyl)-chroman-7-yl]-acetic acid methyl ester and [2-(3-Oxo-propyl)-chroman-5-yl]-acetic acid methyl ester: To a solution of [2-(3-hydroxy-propyl)-chroman-7-yl]-acetic acid methyl ester and [2-(3-hydroxy-propyl)-chroman-5-yl]-acetic acid methyl ester (365 mg, 1.38 mmol) in CH₂Cl₂ (10 mL) was added Dess-Martin solution (15 wt % in CH₂Cl₂) (4.7 mL, 1.66 mmol). The resulting mixture was stirred at room temperature for 1 h. The reaction mixture was diluted with ethyl acetate (50 mL) and washed with water, brine and dried over Na₂SO₄. The solution was concentrated under reduced pressure to give 354 mg (98%) of the desired products which were used in the next step without purification. ¹H NMR (400 MHz, CDCl₃) δ 9.51 (m, 2H), 7.05 (t, 1H), 6.98 (d, 1H), 6.74 (m, 4H), 3.98 (m, 2H), 3.69 (s, 3H), 3.68 (s, 3H), 3.58 (s, 2H), 3.53 (s, 2H), 2.72 (m, 7H), 2.02 (m, 5H), 1.75 (m, 2H), 1.58 (m, 2H).

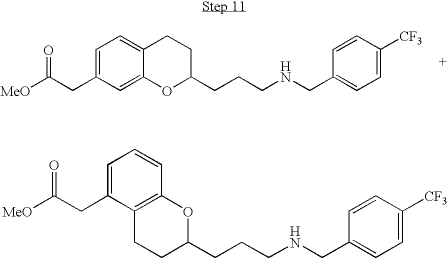

{2-[3-(4-Trifluoromethyl-benzylamino)-propyl]-chroman-7-yl}-acetic acid methyl ester and {2-[3-(4-Trifluoromethyl-benzylamino)-propyl]-chroman-5-yl}-acetic acid methyl ester: To a solution of [2-(3-oxo-propyl)-chroman-7-yl]-acetic acid methyl ester and [2-(3-oxo-propyl)-chroman-5-yl]-acetic acid methyl ester (354 mg, 1.35 mmol) and 4-trifluoromethyl-benzylamine (0.2 mL, 1.35 mmol, 1.0 equiv.) in 3:1 of MeOH/TMOF (trimethylorthoformate) (15 mL) was added NaBH₄ (100 mg, 2.1 mmol) in three portions at 0° C. with stirring. The reaction mixture was stirred at 0° C. for 15 min and then at room temperature for 5 h. The reaction mixture was concentrated under reduced pressure and the residue was dissolved in water (50 mL) and extracted with ethyl acetate (50 mL×2). The combined organic layers were washed with water, brine and dried over Na₂SO₄. The solution was concentrated under reduced pressure and the residue was purified by chromatography to give 291 mg (50%) of the desired compounds. ¹H NMR (400 MHz, CDCl₃) δ 7.58 (d, 4H), 7.45 (d, 4H), 7.05 (t, 1H), 6.98 (d, 1H), 6.74 (m, 4H), 3.98 (m, 2H), 3.87 (s, 4H), 3.69 (s, 3H), 3.68 (s, 3H), 3.58 (s, 2H), 3.53 (s, 2H), 2.70 (m, 8H), 2.00 (m, 2H), 1.72 (m, 8H), 1.52 (m, 2H).

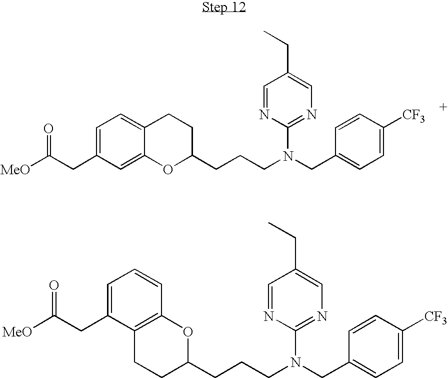

(2-{3-[(5-Ethyl-pyrimidin-2-yl)-(4-trifluoromethyl-benzyl)-amino]-propyl}-chroman-7-yl)-acetic acid methyl ester and (2-{3-[(5-Ethyl-pyrimidin-2-yl)-(4-trifluoromethyl-benzyl)-amino]-propyl}-chroman-5-yl)-acetic acid methyl ester: A solution of {2-[3-(4-trifluoromethyl-benzylamino)-propyl]-chroman-7-yl}-acetic acid methyl ester and {2-[3-(4-trifluoromethyl-benzylamino)-propyl]-chroman-5-yl}-acetic acid methyl ester (210 mg, 0.50 mmol), 2-chloro-5-ethylpyrimidine (0.1 mL, 0.55 mmol) and triethylamine (0.15 mL, 1.0 mmol) in toluene (3 mL) was sealed in a high pressure reaction tube. The reaction mixture was heated to 210° C. in a microwave oven for 3 h. After cooled to room temperature, the mixture was diluted with ethyl acetate (30 mL) and then washed with water, brine and dried over Na₂SO₄. The solution was concentrated under reduced pressure and the residue was purified by chromatography to give the desired products which were separated by preparative TLC (ether/hexanes=1: 2) to give 16.7 mg (12%) of (2-{3-[(5-Ethyl-pyrimidin-2-yl)-(4-trifluoromethyl-benzyl)-amino]-propyl}-chroman-7-yl)-acetic acid methyl ester. ¹H NMR (400 MHz, CDCl₃) δ 8.18 (s, 2H), 7.54 (d, 2H), 7.35 (d, 2H), 6.97 (d, 1H), 6.72 (d, 1H), 6.69 (s, 1H), 4.94 (s, 2H), 3.97 (m, 1H), 3.67 (s, 3H), 3.65 (t, 2H), 3.52 (s, 2H), 2.71 (m, 2H), 2.49 (q, 2H), 1.91 (m, 2H), 1.71 (m, 4H), 1.20 (t, 3H) and 23.8 mg (17%) of (2-{3-[(5-Ethyl-pyrimidin-2-yl)-(4-trifluoromethyl-benzyl)-amino]-propyl}-chroman-5-yl)-acetic acid methyl ester. ¹H NMR (400 MHz, CDCl₃) δ 8.18 (s, 2H), 7.54 (d, 2H), 7.34 (d, 2H), 7.04 (t, 1H), 6.76 (d, 1H), 6.70 (d, 1H), 4.94 (s, 2H), 3.94 (m, 1H), 3.68 (s, 3H), 3.65 (t, 2H), 3.58 (s, 2H), 2.68 (m, 2H), 2.47 (q, 2H), 2.00 (m, 2H), 1.73 (m, 4H), 1.20 (t, 3H).

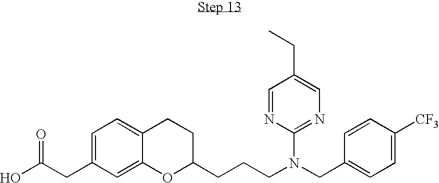

(2-{3-[(5-Ethyl-pyrimidin-2-yl)-(4-trifluoromethyl-benzyl)-amino]-propyl}-chroman-7-yl)-acetic acid: A solution of (2-{3-[(5-ethyl-pyrimidin-2-yl)-(4-trifluoromethyl-benzyl)-amino]-propyl}-chroman-7-yl)-acetic acid methyl ester (16.7 mg, 0.03 mmol) and 1N LiOH (0.1 mL, 0.1 mmol) in 3:1 of THF/MeOH (2 mL) was stirred at room temperature for 3 h. The reaction mixture was concentrated under reduced pressure and the residue was taken up with water (5 mL) and neutralized with 1N HCl (0.1 mL). The resulting mixture was extracted with ethyl acetate (10 mL) and the organic layer was washed with water, brine and then dried over Na₂SO₄. The organic solution was concentrated under reduced pressure to give 13.4 mg (87%) of the title compound. ¹H NMR (400 MHz, CDCl₃) δ 8.19 (s, 2H), 7.53 (d, 2H), 7.34 (d, 2H), 6.97 (d, 1H), 6.73 (d, 1H), 6.69 (s, 1H), 4.94 (s, 2H), 3.96 (m, 1H), 3.64 (t, 2H), 3.55 (s, 2H), 2.76 (m, 2H), 2.47 (q, 2H), 1.90 (m, 2H), 1.69 (m, 4H), 1.20 (t, 3H).

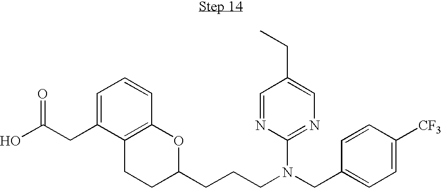

(2-{3-[(5-Ethyl-pyrimidin-2-yl)-(4-trifluoromethyl-benzyl)-amino]-propyl}-chroman-5-yl)-acetic acid: (2-{3-[(5-ethyl-pyrimidin-2-yl)-(4-trifluoromethyl-benzyl)-amino]-propyl}-chroman-5-yl)-acetic acid methyl ester (23.8 mg) was hydrolyzed followed the procedure of step 9 in Example 1 to give 20.4 mg (90%) of the title compound. ¹H NMR (400 MHz, CDCl₃) δ 8.20 (s, 2H), 7.53 (d, 2H), 7.34 (d, 2H), 7.04 (t, 1H), 6.78 (d, 1H), 6.70 (d, 1H), 4.93 (q, 2H), 3.89 (m, 1H), 3.63 (m, 2H), 3.60 (s, 2H), 2.69 (m, 2H), 2.47 (q, 2H), 1.90 (m, 2H), 1.70 (m, 4H), 1.22 (t, 3H).

### EXAMPLE 12

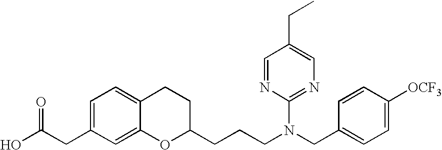

### 2-(2-(3-((5-Ethylpyrimidin-2-yl)(4-(trifluoromethoxy)benzyl)amino)propyl)chroman-7-yl)acetic acid

The title compound was prepared as outlined in Example 10 using 4-trifluoromethoxy-benzylamine. ¹H NMR (400 MHz, CDCl₃) δ 8.19 (s, 2H), 7.26 (d, 2H), 7.13 (d, 2H), 6.98 (d, 1H), 6.73 (d, 1H), 6.70 (s, 1H), 4.88 (s, 2H), 3.95 (m, 1H), 3.62 (t, 2H), 3.54 (s, 2H), 2.74 (m, 2H), 2.47 (q, 2H), 1.85 (m, 2H), 1.68 (m, 4H), 1.20 (t, 3H).

### EXAMPLE 13

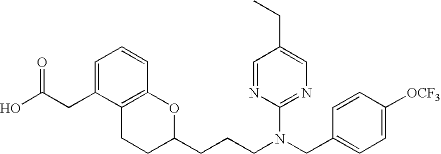

### 2-(2-(3-((5-Ethylpyrimidin-2-yl)(4-(trifluoromethoxy)benzyl)amino)propyl)chroman-5-yl)acetic acid

The title compound was prepared as outlined in Example 11 using 4-trifluoromethoxy-benzylamine. ¹H NMR (400 MHz, CDCl₃) δ 8.22 (s, 2H), 7.25 (d, 2H), 7.13 (d, 2H), 7.04 (t, 1H), 6.78 (d, 1H), 6.71 (d, 1H), 4.88 (q, 2H), 3.87 (m, 1H), 3.60 (s, 2H), 3.59 (m, 2H), 2.68 (m, 2H), 2.47 (q, 2H), 1.85 (m, 2H), 1.65 (m, 4H), 1.22 (t, 3H).

### EXAMPLE 14

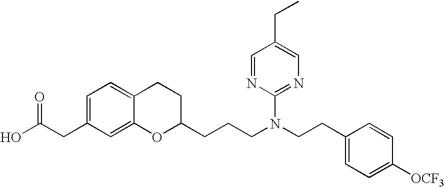

### 2-(2-(3-((5-Ethylpyrimidin-2-yl)(4-(trifluoromethoxy)phenethyl)amino)propyl)chroman-7-yl)acetic acid

The title compound was prepared as outlined in Example 10 using 2-(4-(trifluoromethoxy)phenyl)ethanamine. ¹H NMR (400 MHz, CDCl₃) δ 8.18 (s, 2H), 7.26 (d, 2H), 7.12 (d, 2H), 6.98 (d, 1H), 6.73 (d, 1H), 6.72 (s, 1H), 3.95 (m, 1H), 3.76 (t, 2H), 3.55 (s, 2H), 3.53 (m, 2H), 2.93 (t, 2H), 2.77 (m, 2H), 2.46 (q, 2H), 1.85 (m, 2H), 1.68 (m, 4H), 1.20 (t, 3H).

### EXAMPLE 15

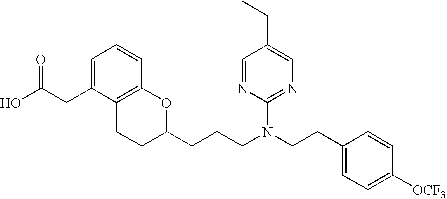

### 2-(2-(3-((5-Ethylpyrimidin-2-yl)(4-(trifluoromethoxy)phenethyl)amino)propyl)chroman-5-yl)acetic acid

The title compound was prepared as outlined in Example 11 using 2-(4-(trifluoromethoxy)phenyl)ethanamine. ¹H NMR (400 MHz, CDCl₃) δ 8.20 (s, 2H), 7.25 (d, 2H), 7.12 (d, 2H), 7.05 (t, 1H), 6.78 (d, 1H), 6.72 (d, 1H), 3.87 (m, 1H), 3.76 (m, 2H), 3.61 (s, 2H), 3.49 (m, 2H), 2.91 (t, 2H), 2.70 (m, 2H), 2.46 (q, 2H), 1.85 (m, 2H), 1.62 (m, 4H), 1.20 (t, 3H).

### EXAMPLE 16

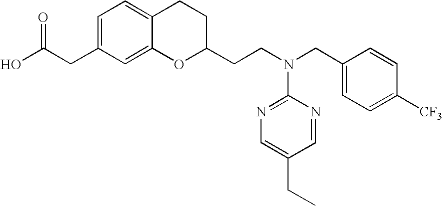

### 2-(2-(2-((5-ethylpyrimidin-2-yl)(4-(trifluoromethyl)benzyl)amino)ethyl)chroman-7-yl)acetic acid

### EXAMPLE 17

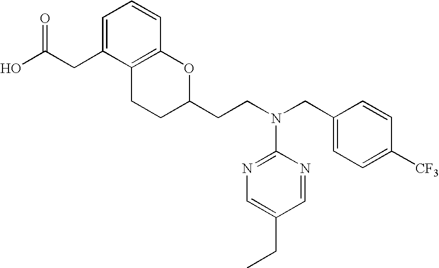

### 2-(2-(2-((5-ethylpyrimidin-2-yl)(4-(trifluoromethyl)benzyl)amino)ethyl)chroman-5-yl)acetic acid

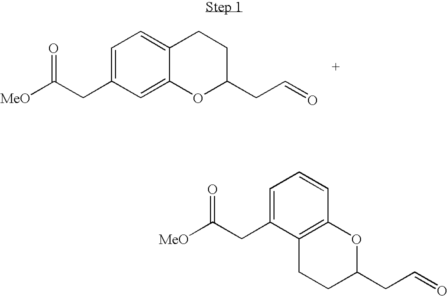

[2-(2-Oxo-ethyl)-chroman-7-yl]-acetic acid methyl ester and [2-(2-Oxo-ethyl)-chroman-5-yl]-acetic acid methyl ester: To a solution of (2-allyl-chroman-7-yl)-acetic acid methyl ester and (2-allyl-chroman-5-yl)-acetic acid methyl ester (523 mg, 2.12 mmol, 1.0 equiv.) in 3:1 dioxane/H₂O (8 mL) was added catalytic amount of OsO₄ (˜5 mg). The resulting mixture was stirred at room temperature for 30 min and the solution became dark purple. NaIO₄ (1.36 g, 6.37 mmol, 3.0 equiv.) was added to the mixture and stirred at room temperature for 2 h. The reaction mixture was diluted with ethyl acetate (50 mL) and washed with water, brine and dried over Na₂SO₄. The solution was concentrated under reduced pressure to give 530 mg (99% yield) of the desired products which were used in next step without purification. ¹H NMR (400 MHz, CDCl₃) δ 9.90 (m, 2H), 7.07 (t, 1H), 7.00 (d, 1H), 6.76 (m, 4H), 4.52 (m, 2H), 3.71 (s, 3H), 3.69(s, 3H), 3.59 (s, 2H), 3.53 (s, 2H), 2.85 (m, 2H), 2.75 (m, 4H), 2.68 (m, 2H), 2.10 (m, 2H), 1.82 (m, 2H).

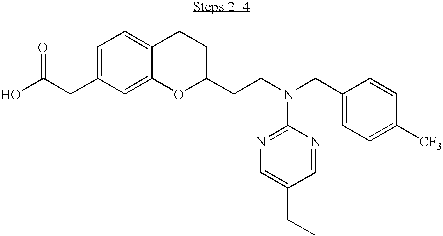

2-(2-(2-((5-Ethylpyrimidin-2-yl)(4-(trifluoromethyl)benzyl)amino)ethyl)chroman-7-yl)acetic acid: The title compound was prepared as outlined in Example 10 using [2-(2-oxo-ethyl)-chroman-7-yl]-acetic acid methyl ester. ¹H NMR (400 MHz, CDCl₃) δ 8.19 (s, 2H), 7.53 (d, 2H), 7.34 (d, 2H), 6.98 (d, 1H), 6.74 (d, 1H), 6.72 (s, 1H), 5.02 (d, 1H), 4.87 (d, 2H), 3.96 (m, 1H), 3.84 (m, 1H), 3.74 (m, 1H), 3.56 (s, 2H), 2.74 (m, 2H), 2.47 (q, 2H), 1.97 (m, 3H), 1.72 (m, 1H), 1.20 (t, 3H).

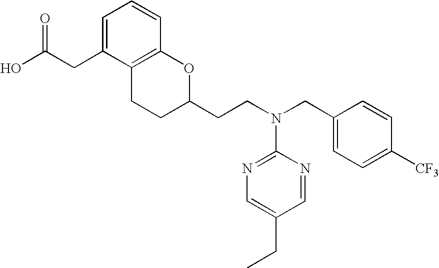

2-(2-(2-((5-Ethylpyrimidin-2-yl)(4-(trifluoromethyl)benzyl)amino)ethyl)chroman-5-yl)acetic acid: The title compound was prepared as outlined in Example 11 using [2-(2-oxo-ethyl)-chroman-5-yl]-acetic acid methyl ester. ¹H NMR (400 MHz, CDCl₃) δ 8.20 (s, 2H), 7.53 (d, 2H), 7.34 (d, 2H), 7.06 (t, 1H), 6.79 (d, 1H), 6.74 (d, 1H), 5.04 (d, 1H), 4.84 (d, 1H), 3.96 (m, 1H), 3.84 (m, 1H), 3.73 (m, 1H), 3.59 (s, 2H), 2.67 (m, 2H), 2.47 (q, 2H), 2.00 (m, 3H), 1.72 (m, 1H), 1.20 (t, 3H).

Note: Absolute stereochemistry was not determined for Examples 18 and 19.

### EXAMPLE 18 STEREOISOMER A

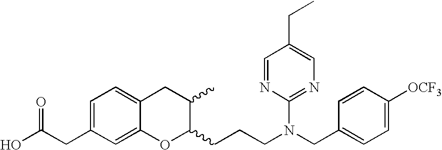

### Cis isomer of 2-(2-(3-((5-ethylpyrimidin-2-yl)(4-(trifluoromethoxy)benzyl)amino)propyl)-3-methylchroman-7-yl)acetic acid

### EXAMPLE 18 STEREOISOMER B

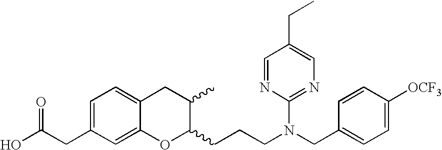

### Cis isomer of 2-(2-(3-((5-ethylpyrimidin-2-yl)(4-(trifluoromethoxy)benzyl)amino)propyl)-3-methyl)chroman-7-yl)acetic acid

### EXAMPLE 18 STEREOISOMER C

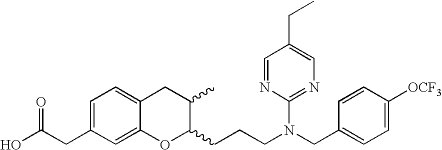

### Trans isomer of 2-(2-(3-((5-ethylpyrimidin-2-yl)(4-(trifluoromethoxy)benzyl)amino)propyl)-3-methyl)chroman-7-yl)acetic acid

### EXAMPLE 18 STEREOISOMER D

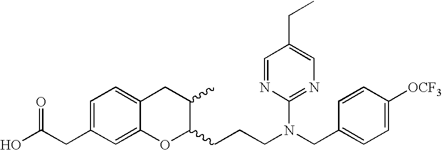

### Trans isomer of 2-(2-(3-((5-ethylpyrimidin-2-yl)(4-(trifluoromethoxy)benzyl)amino)propyl)-3-methyl)chroman-7-yl)acetic acid

### EXAMPLE 19 STEREOISOMER A

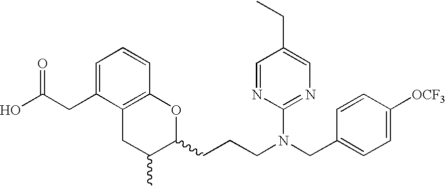

### Cis isomer of 2-(2-(3-((5-ethylpyrimidin-2-yl)(4-(trifluoromethoxy)benzyl)amino)propyl)-3-methylchroman-5-yl)acetic acid

### EXAMPLE 19 STEREOISOMER B

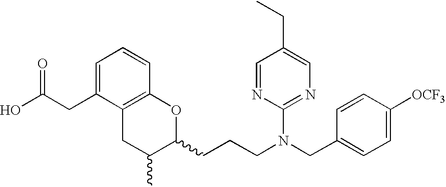

### Cis isomer of 2-(2-(3-((5-ethylpyrimidin-2-yl)(4-(trifluoromethoxy)benzyl)amino)propyl)-3-methylchroman-5-yl)acetic acid

### EXAMPLE 19 STEREOISOMER C

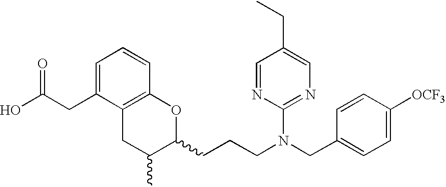

### Trans isomer of 2-(2-(3-((5-ethylpyrimidin-2-yl)(4-(trifluoromethoxy)benzyl)amino)propyl)-3-methylchroman-5-yl)acetic acid

### EXAMPLE 19 STEREOISOMER D

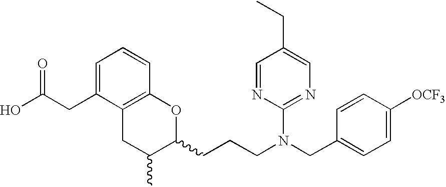

### Trans isomer of 2-(2-(3-((5-ethylpyrimidin-2-yl)(4-(trifluoromethoxy)benzyl)amino)propyl)-3-methylchroman-5-yl)acetic acid

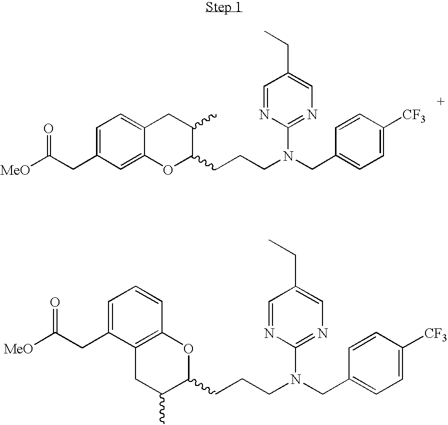

Methyl 2-(2-(3-((5-ethylpyrimidin-2-yl)(4-(trifluoromethyl)benzyl)amino)propyl)-3-methylchroman-7-yl)acetate (two cis enantiomers and two trans enantiomers) and methyl 2-(2-(3-((5-ethylpyrimidin-2-yl)(4-(trifluoromethyl)benzyl)amino)propyl)-3-methylchroman-5-yl)acetate (two cis enantiomers and two trans enantiomers): The title compounds were prepared as outlined in Examples 10 and 11 using 3-bromo-2-methylprop-1-ene. The mixture of eight compounds (each of the two regioisomers has four stereoisomers) was first purified by chromatography and then separated by HPLC on chiral column. The chiral separation was performed on a Dionex LCMS system and two steps were involved. The first separation yielded 4 fractions with each fraction containing two compounds (which were not always a pair of enantiomers). The conditions for the first separation were: Column: Chiral column: Chiralpak AD-H, 10×250 mm, Semi-preparative, Chiral-Tech; Solvent: 98% Hexane with 0.1% TFA/2% Ethanol; Flow rate: 5 mL/min; Inject volume: 50 μL. Run time: 25 min. The retention times for the four fractions were 11.0 min (F1), 15.6 min (F2), 17.9 min (F3) and 21.5 min (F4), respectively. The conditions for the chiral separation of the four fractions; F1, F2, F3 and F4, are described below.

The conditions for F1 (11.0 min) were: Column: Chiralpak OD-H, 4.6×250 mm, Analytical, Chiral-Tech; Solvent: 90% Hexane with 0.1% TFA/10% isopropanol with 0.1% TFA; Flow rate: 1 mL/min; Inject volume: 50 μL. Run time: 10 min. The first fraction (F1-A, RT=6.1 min) yielded:

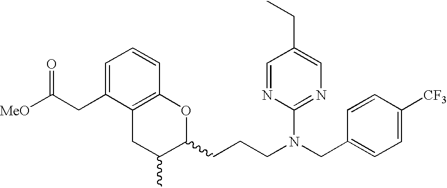

Cis isomer of methyl 2-(2-(3-((5-ethylpyrimidin-2-yl)(4-(trifluoromethyl)benzyl)-amino)propyl)-3-methylehroman-5-yl)acetate: ¹H NMR (400 MHz, CDCl₃) δ 8.44 (s, 2H), 7.26 (d, 2H), 7.16 (d, 2H), 7.04 (t, 1H), 6.76 (d, 1H), 6.68 (d, 1H), 4.95 (q, 2H), 3.68 (s, 3H), 3.67 (m, 3H), 3.57 (s, 2H), 2.71 (m, 1H), 2.57 (q, 2H), 2.29 (m, 1H), 2.11-1.81 (m, 4H), 1.63 (m, 1H), 1.25 (t, 3H), 1.01 (d, 3H). And the second fraction (F1-B, RT=7.5 min) yielded:

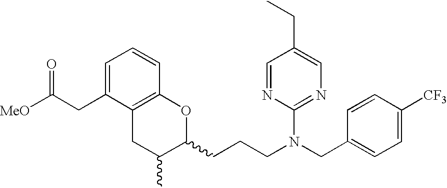

Trans isomer of methyl 2-(2-(3-((5-ethylpyrimidin-2-yl)(4-(trifluoromethyl)benzyl)-amino)propyl)-3-methylehroman-5-yl)acetate: ¹H NMR (400 MHz, CDCl₃) δ 8.43 (s, 2H), 7.26 (d, 2H), 7.16 (d, 2H), 7.04 (t, 1H), 6.77 (d, 1H), 6.67 (d, 1H), 5.03 (d, 1H), 4.84 (d, 1H), 3.99 (m, 1H), 3.68 (s, 3H), 3.67 (m, 2H), 3.57 (s, 2H), 2.80 (dd, 1H), 2.57 (q, 2H), 2.40 (m, 1H), 2.13 (m, 1H), 1.88 (m, 1H), 1.80 (m, 1H), 1.64 (m, 1H), 1.57 (m, 1H), 1.26 (t, 3H), 0.93 (d, 3H).

The conditions for F2 (15.6 min) were: Column: Chiralpak OD-H, 4.6×250 mm, Analytical, Chiral-Tech; Solvent: 97% Hexane with 0.1% TFA/3°% isopropanol with 0.1% TFA; Flow rate: 1 mL/min; Inject volume: 50 μL. Run time: 15 min. The first fraction (F2-A, R_{T}=9.0 min) yielded:

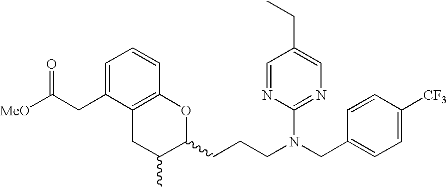

Cis isomer of methyl 2-(2-(3-((5-ethylpyrimidin-2-yl)(4-(trifluoromethyl)benzyl)-amino)propyl)-3-methylehroman-5-yl)acetate: This is the enantiomer of F1-A and has identical H NMR as F1-A. And the second fraction (F2-B, RT=11.8 min) yielded:

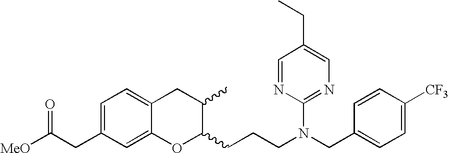

Cis isomer of methyl 2-(2-(3-((5-ethylpyrimidin-2-yl)(4-(trifluoromethyl)benzyl)-amino)propyl)-3-methylehroman-7-yl)acetate: ¹H NMR (400 MHz, CDCl₃) δ 8.41 (s, 2H), 7.27 (d, 2H), 7.16 (d, 2H), 6.96 (d, 1H), 6.73 (d, 1H), 6.67 (s, 1H), 4.95 (q, 2H), 3.68 (s, 3H), 3.67 (m, 3H), 3.53 (s, 2H), 2.72 (dd, 1H), 2.55 (q, 2H), 2.42 (dd, 1H), 2.23 (m, 1H), 1.97 (m, 1H), 1.83 (m, 2H), 1.57 (m, 1H), 1.25 (t, 3H), 0.98 (d, 3H).

The conditions for F3 (17.9 min) were: Column: Chiralpak AD-H, 4.6×250 mm, Analytical, Chiral-Tech; Solvent: 97% Hexane with 0.1% TFA/3°% isopropanol with 0.1% TFA; Flow rate: 1 mL/min; Inject volume: 50 μL. Run time: 21 min. The first fraction (F3-A, RT=15.8 min) yielded:

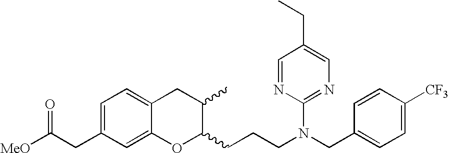

Trans isomer of methyl 2-(2-(3-((5-ethylpyrimidin-2-yl)(4-(trifluoromethyl)benzyl)-amino)propyl)-3-methylchroman-7-yl)acetate: ¹H NMR (400 MHz, CDCl₃) δ 8.39 (s, 2H), 7.26 (d, 2H), 7.16 (d, 2H), 6.96 (d, 1H), 6.74 (d, 1H), 6.67 (s, 1H), 5.02 (d, 1H), 4.85 (d, 1H), 3.99 (m, 1H), 3.69 (m, 1H), 3.68 (s, 3H), 3.60 (m, 1H), 3.57 (s, 2H), 2.92 (dd, 1H), 2.55 (q, 2H), 2.43 (dd, 1H), 2.07 (m, 2H), 1.98-1.55 (m, 3H), 1.22 (t, 3H), 0.91 (d, 3H).

And the second fraction (F3-B, RT=17.9 min) yielded:

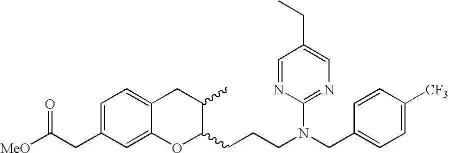

Cis isomer of methyl 2-(2-(3-((5-ethylpyrimidin-2-yl)(4-(trifluoromethyl)benzyl)-amino)propyl)-3-methylchroman-7-yl)acetate: This is the enantiomer of F2-B and has identical ¹H NMR as F2-B.

The conditions for F4 (21.5 min) were: Column: Chiralpak IA-H, 4.6×250 mm, Analytical, Chiral-Tech; Solvent: 90% Hexane with 0.1% TFA/10% isopropanol with 0.1% TFA; Flow rate: 1 mL/min; Inject volume: 50 μL. Run time: 20 min. The first fraction (F4-A, RT=8.9 min) yielded:

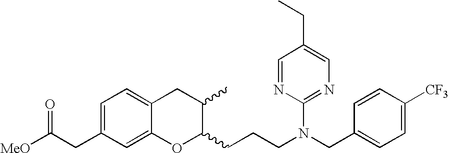

Trans isomer of methyl 2-(2-(3-((5-ethylpyrimidin-2-yl)(4-(trifluoromethyl)benzyl)-amino)propyl)-3-methylehroman-7-yl)acetate: This is the enantiomer of F3-A and has identical ¹H NMR as F3-A. And the second fraction (F4-B, RT=16.9 min) yielded:

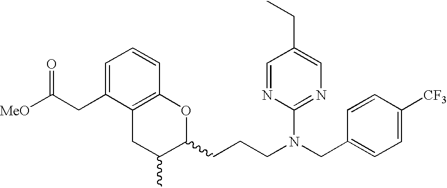

Trans isomer of methyl 2-(2-(3-((5-ethylpyrimidin-2-yl)(4-(trifluoromethyl)benzyl)-amino)propyl)-3-methylehroman-5-yl)acetate: This is the enantiomer of F1-B and has identical H NMR as F1-B.

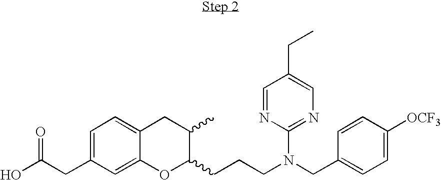

### EXAMPLE 18A

Cis isomer of 2-(2-(3-((5-ethylpyrimidin-2-yl)(4-(trifluoromethyl)benzyl)amino)-propyl)-3-methylehroman-7-yl)acetic acid: The compound was prepared from F2-B following the procedure of Step 13 in Examples 10 and 11. ¹H NMR (400 MHz, CDCl₃) δ 8.18 (s, 2H), 7.26 (d, 2H), 7.12 (d, 2H), 6.96 (d, 1H), 6.73 (d, 1H), 6.70 (s, 1H), 4.88(s, 2H), 3.64 (m, 3H), 3.56 (s, 2H), 2.72 (dd, 1H), 2.47 (q, 2H), 2.41 (dd, 1H), 2.01-1.40 (m, 5H), 1.20 (t, 3H), 0.97 (d, 3H).

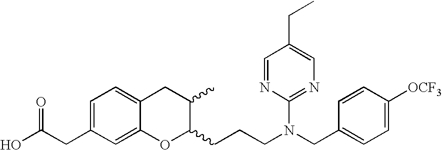

### EXAMPLE 18B

Cis isomer of 2-(2-(3-((5-ethylpyrimidin-2-yl)(4-(trifluoromethoxy)benzyl)amino)-propyl)-3-methyl)chroman-7-yl)acetic acid: The compound was prepared from F3-B following the procedure of Step 13 in Examples 10 and 11. The compound is the enantiomer of Example 18A and therefore has the identical ¹H NMR spectra.

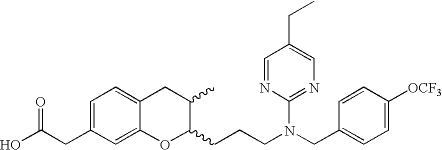

### EXAMPLE 18C

Trans isomer of 2-(2-(3-((5-ethylpyrimidin-2-yl)(4-(trifluoromethyl)benzyl)amino)-propyl)-3-methylehroman-7-yl)acetic acid: The compound was prepared from F3-A following the procedure of Step 13 in Examples 10 & 11. ¹H NMR (400 MHz, CDCl₃) δ 8.18 (s, 2H), 7.25 (d, 2H), 7.13 (d, 2H), 6.98 (d, 1H), 6.76 (d, 1H), 6.70 (s, 1H), 4.87 (s, 2H), 3.98 (m, 1H), 3.62 (t, 2H), 3.57 (s, 2H), 2.92 (dd, 1H), 2.45 (q, 2H), 2.43 (dd, 1H), 2.07-1.55 (m, 5H), 1.20 (t, 3H), 0.96 (d, 3H).

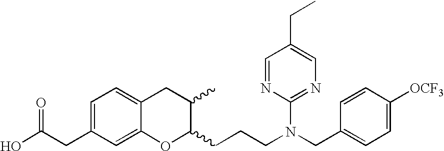

### EXAMPLE 18D

Trans isomer of 2-(2-(3-((5-ethylpyrimidin-2-yl)(4-(trifluoromethoxy)benzyl)amino)-propyl)-3-methyl)chroman-7-yl)acetic acid: The compound was prepared from F4-A following the procedure of Step 13 in Examples 10 & 11. The compound is the enantiomer of Example 18C and therefore has the identical ¹H NMR spectra.

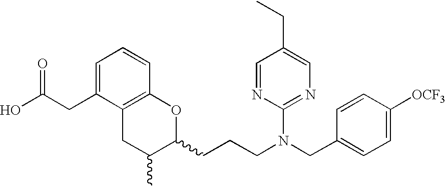

### EXAMPLE 19A

Cis isomer of 2-(2-(3-((5-ethylpyrimidin-2-yl)(4-(trifluoromethoxy)benzyl)amino)-propyl)-3-methylehroman-5-yl)acetic acid: The compound was prepared from F1-A following the procedure of Step 14 in Examples 10 & 11. ¹H NMR (400 MHz, CDCl₃) δ 8.18 (s, 2H), 7.26 (d, 2H), 7.12 (d, 2H), 7.06 (t, 1H), 6.78 (d, 1H), 6.72 (d, 1H), 4.88 (s, 2H), 3.63 (m, 3H), 3.60 (s, 2H), 2.72 (dd, 1H), 2.47 (q, 2H), 2.30 (dd, 1H), 2.11-1.45 (m, 5H), 1.20 (t, 3H), 0.99 (d, 3H).

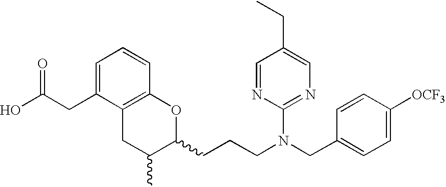

### EXAMPLE 19B

Cis isomer of 2-(2-(3-((5-ethylpyrimidin-2-yl)(4-(trifluoromethoxy)benzyl)amino)-propyl)-3-methylchroman-5-yl)acetic acid: The compound was prepared from F2-A following the procedure of Step 14 in Examples 10 & 11. The compound is the enantiomer of Example 19A and therefore has the identical ¹H NMR spectra.

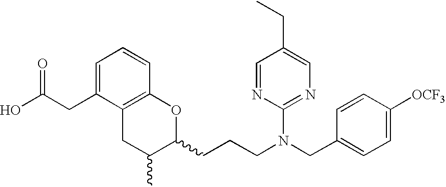

### EXAMPLE 19C

Trans isomer of 2-(2-(3-((5-ethylpyrimidin-2-yl)(4-(trifluoromethyl)benzyl)amino)-propyl)-3-methylchroman-5-yl)acetic acid: The compound was prepared from F1-B following the procedure of Step 14 in Examples 10 & 11. ¹H NMR (400 MHz, CDCl₃) δ 8.18 (s, 2H), 7.24 (d, 2H), 7.12 (d, 2H), 7.05 (t, 1H), 6.78 (d, 1H), 6.71 (d, 1H), 4.86 (s, 2H), 3.97 (m, 1H), 3.60 (s, 2H), 3.60 (t, 2H), 2.81 (dd, 1H), 2.47 (q, 2H), 2.42 (dd, 1H), 2.11 (m, 1H), 1.88 (m, 1H), 1.80 (m, 2H), 1.44 (m, 1H), 1.20 (t, 3H), 0.91 (d, 3H).

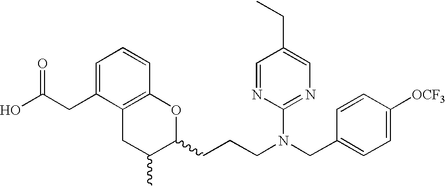

### EXAMPLE 19D

Trans isomer of 2-(2-(3-((5-ethylpyrimidin-2-yl)(4-(trifluoromethoxy)benzyl)amino)-propyl)-3-methylchroman-5-yl)acetic acid: The compound was prepared from F4-B following the procedure of Step 14 in Examples 10 & 11. The compound is the enantiomer of Example 19C and therefore has the identical ¹H NMR spectra.

### Biological Activity Assay

### Full-Length Human and Rhesus PPAR Transcriptional Activation Assay

HEK293 cells were seeded the day before transfection into 384-well plates at a cell density of 6,000 cells per well in 40 μl assay medium I (phenol red-free DMEM containing 4% charcoal-dextran stripped FBS, 1% Penicillin-Streptomycin and 1% GlutaMax-1). Then 25 ng of PPRE::Luciferase reporter plasmid, an expression plasmid (25 pg of PPARα, or 40 pg of PPARδ, or 75 pg of PPARγ2), and an appropriate amount of the plasmid pUC19 to bring the total DNA amount to 50 ng was added to 20 μl of phenol-red free DMEM and 150 nL of Fugene 6 and incubated for 30 minutes. The transfection mixtures were then added to the cells and incubated for three hours. 10 μl of test agents in 5% DMSO were then added to the cells and incubated at 37° C. for an additional 18 hours. Luciferase activity was then assayed by adding 25 μl/well of Britelite (Perkin Elmer) according to the manufacturer's protocol and relative light output was measured with an Analyst GT plate reader (Molecular Devices). All experimental points were done in triplicate and the assays were repeated at least 3 times.

### Full-Length Mouse PPAR Transcriptional Activation Assay

HEK293 cells were seeded the day before transfection in 15 cm² dishes at a density of 9×10⁶ cells/dish and incubated at 37° C., 10% CO₂ for 16-24 hours. Then, 4.5 pg of PPRE::Luciferase reporter plasmid, an expression plasmid (7.5 ng of PPARα, or 7.5 ng of PPARδ, or 75 ng of PPARγ2), and an appropriate amount of the plasmid pUC19 to bring the total DNA amount to 18 pg were mixed with 54 μl of Fugene 6 in 2 mls of phenol red-free DMEM and incubated for 30 minutes. Transfection mixtures were then incubated with the cells for 18 hours. Cells were then replated into sterile, white TC treated 384-well assay plates at a cell density of 24×10³ cells/well in 40 μl of assay medium II (phenol-red free DMEM containing 3% charcoal/dextran-stripped FBS, 1% Penicillin-Streptomycin and 1% GlutaMax-1) and incubated at 37° C. for 6 hours. 10 μl of test agents in 5% DMSO were then added to the cells and incubated at 37° C. for an additional 18 hours. Luciferase activity was then assayed by adding 30 μl/well of Britelite (Perkin Elmer) according to the manufacturer's protocol and relative light output was measured with an Analyst GT plate reader (Molecular Devices). All experimental points were done in triplicate and the assays were repeated 3 times.

TABLE 1
Biological Activity

| | PPAR alpha | PPAR delta | PPAR gamma |
|---|---|---|---|
| Ex- | + indicates ≦1 μM | + indicates ≦1 μM | + indicates ≦1 μM |
| ample # | − indicates >1 μM | − indicates >1 μM | − indicates >1 μM |
| 1 | − | + | + |
| 2 | − | + | + |
| 3 | − | + | + |
| 4 | − | + | + |
| 5 | − | − | − |
| 6 | − | − | − |
| 7 | − | − | − |
| 8 | − | + | − |
| 9 | − | + | − |
| 10 | − | + | + |
| 11 | − | + | + |
| 12 | − | + | + |
| 13 | − | + | + |
| 14 | − | + | + |
| 15 | − | + | + |
| 16 | + | + | + |
| 17 | + | + | + |
| 18A | − | + | + |
| 18B | + | + | + |
| 18C | + | + | − |
| 18D | − | + | + |
| 19A | − | + | + |
| 19B | − | + | + |
| 19C | − | + | + |
| 19D | − | + | + |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of modulating PPAR comprising the administration of a compound of Formula I 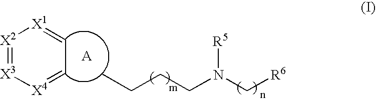
or a salt, ester, or prodrug thereof, wherein:
A is selected from the group consisting of cycloalkyl and heterocycloalkyl, either of which may be optionally substituted;
X¹ is selected from the group consisting of CR¹ and N;
X² is selected from the group consisting of CR² and N;
X³ is selected from the group consisting of CR³ and N;
X⁴ is selected from the group consisting of CR⁴ and N; or any two of X¹, X², X³ and X⁴ may combine to form aryl, cycloalkyl or heterocycloalkyl, any of which may be optionally substituted;
m is 0, 1 or 2;
n is 0, 1, 2 or 3;
R¹-R⁴ are independently selected from the group consisting of alkoxy, alkyl, aryl, arylalkyl, carboxyalkyl, cycloalkyl, esteralkyl, halo, haloalkyl, heteroarylalkyl, heterocycloalkyl, heterocycloalkylalkyl and hydrogen, any of which may be optionally substituted; or, alternatively, any two of R¹, R², R³, and R⁴ may combine to form aryl, cycloalkyl and heterocycloalkyl, which may be optionally substituted; and
R⁵ and R⁶ are independently selected from the group consisting of acyl, alkyl, alkoxy, alkoxyalkyl, alkylene, alkynyl, amido, amino, aminosulfonyl, aryl, arylalkoxy, arylamino, arylthio, carboxy, cycloalkyl, ester, ether, halo, haloalkyl, heteroaryl, heteroarylamino, heterocycloalkyl, hydrazinyl, imino, thio, sulfonate and sulfonyl, any of which may be optionally substituted.

2. A method of treatment of a PPAR-mediated disease comprising the administration of a therapeutically effective amount of a compound as recited in claim 1 to a patient in need thereof.

3. The method as recited in claim 2 wherein said disease is dyslipidemia, metabolic syndrome X, heart failure, hypercholesteremia, cardiovascular disease, type II diabetes mellitus, type 1 diabetes, insulin resistance hyperlipidemia, obesity, anorexia bulimia, hair growth abnormalities, anorexia nervosa, inflammatory diseases, asthma, psoriasis, ulcerative colitis, and dermatitis.

4. A compound of Formula II: 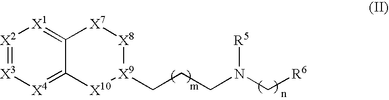
or a salt, ester, or prodrug thereof, wherein:
X¹ is selected from the group consisting of CR¹ and N;
X² is selected from the group consisting of CR² and N;
X³ is selected from the group consisting of CR³ and N;
X⁴ is selected from the group consisting of CR⁴ and N;
X⁷ is selected from the group consisting of C(O), CR^{7a}R^{7b}, O, NR⁷ and S(O)_{g};
X⁸ is selected from the group consisting of C(O), CR^{8a}R^{8b}, O, NR⁸ and S(O)_{g};
X⁹ is selected from the group consisting of CR^{9a} and N;
X¹⁰ is selected from the group consisting of C(O), CR^{10a}R^{10b}, O, NR¹⁰ and S(O)_{g};
m is 0, 1 or 2;
n is 0, 1, 2 or 3;
g is 0, 1 or 2;
R⁵ and R⁶ are independently selected from the group consisting of aryl, and heteroaryl, any of which may be optionally substituted;
R¹-R⁴ are independently selected from the group consisting of alkoxy, alkyl, alkylcarboxy, alkylester, alkylaryl, amido, carboxy, carboxyalkyl, halo, heteroaryl, heteroarylalkyl, heterocycloalkyl and hydrogen, any of which may be optionally substituted;
R^{7a}-R^{10a} and R^{7b}-R^{10b} are independently selected from the group consisting of alkoxy, alkyl, aryl, alkylaryl, carboxy, cycloalkyl, cyano, ester, halo, haloalkyl, heteroarylalkyl, heterocycloalkyl, hydrogen and hydroxyl, any of which may be optionally substituted; and
R⁷-R¹⁰ are independently selected from the group consisting of alkyl, alkylaryl, aryl, cycloalkyl, halo, haloalkyl, heteroaryl, heterocycloalkyl and hydrogen, any of which may be optionally substituted.

5. The compound as recited in claim 4, having structural Formula III: 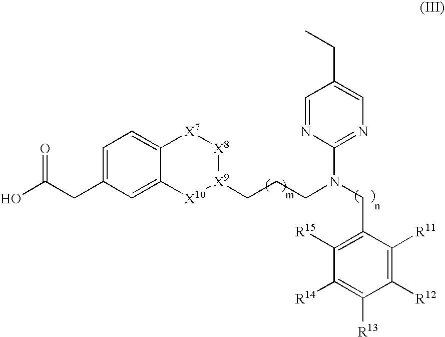
or a salt, ester, or prodrug thereof, wherein:
X⁷ is selected from the group consisting of CR^{7a}R^{7b}, O, and NR⁷;
X⁸ is selected from the group consisting of CR^{8a}R^{8b}, O, and NR⁸;
X⁹ is selected from the group consisting of CR^{9a} and N;
X¹⁰ is selected from the group consisting of CR^{10a}R^{10b}, O, and NR¹⁰;
m is 0, 1 or 2;
n is 0, 1 or 2;
R^{7a}-R^{10a} and R^{7b}-R^{10b} are independently selected from the group consisting of alkoxy, alkyl halo, hydrogen and hydroxyl, any of which may be optionally substituted;
R⁷-R¹⁰ are independently selected from the group consisting of alkyl haloalkyl, hydrogen and null, any of which may be optionally substituted; and
R¹¹, R¹², R¹³, R¹⁴ and R¹⁵ are independently selected from the group consisting of alkoxy, alkyl halo, haloalkyl and hydrogen, any of which may be optionally substituted.

6. The compound as recited in claim 5, or a salt, ester, or prodrug thereof, wherein
X⁷ is CR^{7a}R^{7b}; and
X⁸ is CR^{8a}R^{8b}.

7. The compound as recited in claim 6, or a salt, ester, or prodrug thereof, wherein
X⁷ and X⁸ are each CH₂;
X⁹ is selected from the group consisting of CH or N;
X¹⁰ is selected from the group consisting of CH₂ or O; and
R¹¹-R¹⁵ are independently selected from the group consisting of alkoxy, alkyl halo, haloalkyl and hydrogen, any of which may be optionally substituted.

8. The compound as recited in claim 7, or a salt, ester, or prodrug thereof, wherein
X⁹ is N; and
X¹⁰ is CH₂.

9. The compound as recited in claim 8, or a salt, ester, or prodrug thereof, wherein
R¹³ is selected from the group consisting of trifluoromethyl and trifluoromethoxy; and
R¹¹, R¹², R¹⁴, and R¹⁵ are hydrogen.

10. The compound as recited in claim 7, or a salt, ester, or prodrug thereof, wherein
X⁹ is CH; and
X¹⁰ is O.

11. The compound as recited in claim 10, or a salt, ester, or prodrug thereof, wherein
R¹³ is selected from the group consisting of trifluoromethyl and trifluoromethoxy; and
R¹¹, R¹², R¹⁴, and R¹⁵ are hydrogen.

12. The compound as recited in claim 4, wherein the compound has the Formula V 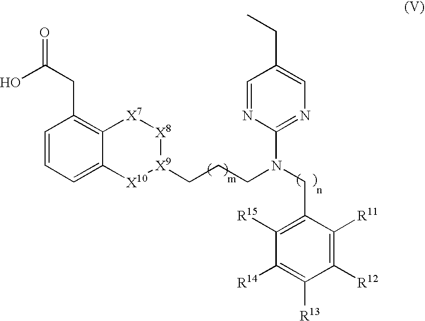
or a salt, ester, or prodrug thereof, wherein:
X⁷ is selected from the group consisting of CR^{7a}R^{7b}, O, and NR⁷;
X⁸ is selected from the group consisting of CR^{8a}R^{8b}, O, and NR⁸;
X⁹ is selected from the group consisting of CR^{9a} and N;
X¹⁰ is selected from the group consisting of CR^{10a}R^{10b}, O, and NR¹⁰;
m is 0, 1 or 2;
n is 0, 1 or 2;
R^{7a}-R^{10a} and R^{7b}-R^{10b} are independently selected from the group consisting of alkoxy, alkyl, halo, hydrogen and hydroxyl, any of which may be optionally substituted;
R⁷-R¹⁰ are independently selected from the group consisting of alkyl, haloalkyl, hydrogen and null, any of which may be optionally substituted; and
R¹¹, R¹², R¹³, R¹⁴ and R¹⁵ are independently selected from the group consisting of alkoxy, alkyl, halo, haloalkyl and hydrogen, any of which may be optionally substituted.

13. The compound as recited in claim 12, or a salt, ester, or prodrug thereof, wherein
X⁷ is CR^{7a}R^{7b}; and
X⁹ is CR^{9a}.

14. The compound as recited in claim 13, or a salt, ester, or prodrug thereof, wherein
X⁷ is CH₂;
X⁹ is CH;
X⁸ is selected from the group consisting of CH₂ and O;
X¹⁰ is selected from the group consisting of CH₂ and O; and
R¹¹-R¹⁵ are independently selected from the group consisting of alkoxy, alkyl halo, haloalkyl and hydrogen, any of which may be optionally substituted.

15. The compound as recited in claim 14, or a salt, ester, or prodrug thereof, wherein
X⁸ is O; and
X¹⁰ is CH₂.

16. The compound as recited in claim 14, or a salt, ester, or prodrug thereof, wherein
X⁸ is CH₂; and
X¹⁰ is O.

17. The compound as recited in claim 16, or a salt, ester, or prodrug thereof, wherein
R¹³ is selected from the group consisting of trifluoromethyl and trifluoromethoxy; and
R¹¹, R¹², R¹⁴, and R¹⁵ are hydrogen.

18. The compound as recited in claim 4 selected from the group consisting of Examples 1-17, 18a 18d, and 19a 19d.

19. A compound as recited in claim 4 for use in the manufacture of a medicament for the prevention or treatment of a disease or condition ameliorated by the modulation of PPAR.

20. A pharmaceutical composition comprising a compound as recited in claim 4 together with a pharmaceutically acceptable carrier.
